(19) United States
(12) Patent Application Publication
(10) Pub. No.: US 2011/0165681 A1
(43) Pub. Date: Jul. 7, 2011

(54) Light-Activated Proton Pumps and Applications Thereof

(75) Inventors: Edward S. Boyden, Chestnut Hill, MA (US); Brian Y. Chow, Cambridge, MA (US); Xue Han, Chestnut Hill, MA (US); Xiaofeng Qian, Burnaby (CA); Nathan Cao Klapoetke, Cambridge, MA (US); Albert Hyukjae Kwon, Cambridge, MA (US)

(73) Assignee: Massachusetts Institute of Technology, Cambridge, MA (US)

(21) Appl. No.: 12/714,436

(22) Filed: Feb. 26, 2010

Related U.S. Application Data
(60) Provisional application No. 61/155,855, filed on Feb. 26, 2009.

(51) Int. Cl. C12N 15/85

(52) U.S. Cl. 435/455

(57) ABSTRACT

In a method for adjusting the voltage potential or pH of, or cause proton release from, cells, subcellular regions, or extracellular regions, a gene encoding for a light-driven proton pump is incorporated into at least one target cell or region, the proton pump operating in response to a specific wavelength of light. Expression of the gene is caused by exposing the target cell or region to the specific wavelength of light in a manner designed to cause the voltage potential adjustment, pH adjustment, or proton release. The proton pump may be a microbial rhodopsin, in particular derived from the halorubrum genus of archaeabacteria, or be derived from leptosphaeria maculans, P. triticirepentis, and S. scelorotorium. The voltage potential of the target cell or region may adjusted until it is hyperpolarized in order to achieve neural silencing. Light-activated proton pumps responsive to different wavelengths of light may be used together to achieve multi-color neural silencing.

### RELATED APPLICATIONS

This application claims the benefit of U.S. Provisional Application Ser. No. 61/155,855, filed Feb. 26, 2009, the entire disclosure of which is herein incorporated by reference.

### STATEMENT REGARDING FEDERALLY SPONSORED RESEARCH OR DEVELOPMENT

This invention was made with U.S. government support under Grant Number NIH DP2-OD002002, awarded by the National Institutes of Health (NIH). The government has certain rights in this invention.

### FIELD OF THE TECHNOLOGY

The present invention relates to methods and devices for control of cell function and, in particular, to light-activated proton pumps and their use for control of transmembrane potential and pH.

### BACKGROUND

Many diseases of the human brain and nervous system are related to dysfunction of specific neuron types, which undergo pathological changes in number, excitability, anatomy, or synaptic connectivity. These changes lead, via altered neural circuit activity, to the perceptual, cognitive, emotional, and motor deficits associated with various neurological and psychiatric illnesses. The ability to optically activate or inactivate genetically-specified excitable target cells, such as central nervous system neurons, glia, peripheral neurons, skeletal muscle, smooth muscle, cardiac muscle, pancreatic islet cells, thymus cells, immune cells, or other excitable cells, embedded in intact tissue, such as brain, peripheral nervous system, muscle, and skin, would enable radical new treatments for many disorders (e.g., neuropathic pain, Parkinson's disease, epilepsy, diabetes, and other diseases).

Molecular-genetic methods for making cells such as neurons sensitive to being activated (e.g., depolarized) or inactivated (e.g., hyperpolarized) by light have been previously developed [see, for example, X. Han and E. S. Boyden, “Multiple-color optical activation, silencing, and desynchronization of neural activity, with single-spike temporal resolution,” PLoS ONE 2, e299 (2007)]. The light-activated cation channel channelrhodopsin-2 (ChR2), and the light-activated chloride pump halorhodopsin (Halo/NpHR), when transgenically expressed in neurons, make them sensitive to being activated by blue light, and silenced by yellow light, respectively [Han, X. and E. S. Boyden (2007). “Multiple-color optical activation, silencing, and desynchronization of neural activity, with single-spike temporal resolution.” PLoS ONE 2(3): e299; Boyden, E. S., F. Zhang, E. Bamberg, G. Nagel and K. Deisseroth (2005). “Millisecond-timescale, genetically targeted optical control of neural activity.” Nat Neurosci 8(9): 1263-8]. ChR2 has been successfully employed in the nonhuman primate brain for optical control of neural dynamics [Han, X., X. Qian, J. Bernstein, H. Zhou, G. Franzesi, P. Stern, R. Bronson, A. Graybiel, R. Desimone, and E. Boyden, “Millisecond Timescale Optical Control of Neurodynamics in the Nonhuman Primate Brain, Neuron 62, 191-198, Apr. 20, 2009].

Bacteriorhodopsins are light-activated electrogenic proton pumps that are 7-transmembrane helix proteins (7-TM), utilize all-trans retinal as their chromophore in their native state, and bear structural similarity to the H. salinarum bacteriorhodopsin. Upon absorption of a photon, the all-trans retinal isomerizes to 13-cis retinal, and typically thermally relaxes to its active all-trans form in the dark (although this process can be facilitated by light). The cis-trans isomerization sets off several coupled structural rearrangements within the molecule that accommodate the active pumping of ions and in some cases, their passive conduction. Their structure-function relationships have been widely studied in model systems, namely intact e. coli, membranes and vesicles reconstituted from e. coli, and xenopus laevis oocytes. Commonly characterized bacteriorhodopsins are the H. salinarum bacteriorhodopsin, the S. ruber xanthorhodopsin, and uncultured gamma-protobacterium BAC31A8. To date, their use has been proposed or demonstrated in the photovoltaic devices, memory storage devices, and the solar powering of single-cellular microbes.

Light-activated and light-gated electrogenic membrane proteins, such as the C. rheinhardtii channelrhodopsin-2 (inwardly rectifying cation channel) and N. pharaonis halorhodopsin (inwardly rectifying chloride pump) have been demonstrated as capable of generating sufficient photocurrents for altering neural activity. However, the use of bacteriorhodopsin-mediated photocurrents to explicitly alter the physiology of multi-cellular organisms, cells and tissue extracted from multi-cellular organisms, and cell lines derived from multi-cellular organisms, such as mammals, has yet to be demonstrated. It has been, to date, unclear whether bacteriorhodopsins could safely generate sufficient photocurrents to alter the physiology in such heterologously expressed systems on account of a variety of factors, such as intracellular trafficking, low ion carrier concentration (i.e. protons, typically, ˜100 nanomolar at physiological pH), and efficacy. In particular, protons are not viewed as primary ions involved in the electrophysiology of excitable cells, such as neurons, and demonstrations of electrogenic activity have not yet been shown in excitable cells.

### SUMMARY

The present invention employs light-activated proton pumps to modify cell parameters, including transmembrane potential and/or pH of cells, their sub-cellular regions, and their local environment. In particular, the use of outwardly rectifying proton pumps can hyperpolarize cells by moving positively charged ions from the cytoplasm to the extracellular environment. Under specific conditions, their use can increase the intracellular pH or decrease the extracellular pH.

In one aspect, the present invention shows that members of the class of light-driven outward proton pumps can mediate very powerful, safe, multiple-color silencing of neural activity. The gene archaerhodopsin-3 (“Arch”) from Halorubrum sodomense enables near-100% silencing of neurons in the awake brain when virally expressed in mouse cortex and illuminated with yellow light. Arch mediates currents of several hundred picoamps at low light powers, and supports neural silencing currents approaching 900 pA at light powers easily achievable in vivo. In addition, Arch spontaneously recovers from light-dependent inactivation, unlike light-driven chloride pumps that enter long-lasting inactive states in response to light. These properties of Arch are appropriate to mediate the optical silencing of significant brain volumes over behaviourally-relevant timescales. Arch function in neurons is well tolerated because pH excursions created by Arch illumination are minimized by self-limiting mechanisms to levels comparable to those mediated by channelrhodopsins or natural spike firing.

In another aspect of the present invention, the blue-green light-drivable proton pump from the fungus Leptosphaeria maculans⁴ (“Mac”) can, when expressed in neurons, enable neural silencing by blue light, thus enabling, alongside other developed reagents, the potential for independent silencing of two neural populations by blue vs. red light. Light-driven proton pumps thus represent a high-performance and extremely versatile class of “optogenetic” voltage and ion modulator, which will broadly empower new neuroscientific, biological, neurological, and psychiatric investigations.

Arch and Mac represent members of a new, diverse, and powerful class of optical neural silencing reagent, the light-driven proton pump, which operates without the need for exogenous chemical supplementation in mammalian cells. The efficacy of these proton pumps is surprising, given that protons occur, in mammalian tissue, at a millionfold-lower concentration than the ions carried by other optical control molecules. This high efficacy may be due to the fast photocycle of Arch, but it may also be due to the ability of high-pKa residues in proton pumps to mediate proton uptake. Several facts were discovered about this class of molecules that point the way for future neuroengineering innovation. First, proton pumping is a self-limiting process in neurons, providing for a safe and naturalistic form of neural silencing. Second, proton pumps recover spontaneously after optical activation, improving their relevance for behaviourally-relevant silencing over the class of halorhodopsins. Finally, proton pumps exist with a wide diversity of action spectra, thus enabling multiple-color silencing of distinct neural populations. Structure-guided mutagenesis of Arch and Mac may further facilitate development of neural silencers with altered spectrum or ion selectivity, given the significant amount of structure-function knowledge of the proton pump family. These opsins are likely to find uses across the spectrum of neuroscientific, biological, and bioengineering fields. For example, expression of these opsins in neurons, muscle, immune cells, and other excitable cells will allow control over their membrane potential, opening up the ability to investigate the causal role of specific cells' activities in intact organisms, and opening up the ability to understand the causal contribution of such cells to disease states in animal models.

In one aspect, the present invention is a method for adjusting the voltage potential of cells, subcellular regions, or extracellular regions, comprising incorporating at least one gene encoding for a light-driven proton pump into at least one target cell, subcellular region, or extracellular region, the proton pump operating to change transmembrane potential in response to a specific wavelength of light, and causing the expression of the gene by exposing the target cell, subcellular region, or extracellular region to the specific wavelength of light in a manner designed to cause the voltage potential of the target cell, subcellular region, or extracellular region to increase or decrease. In a preferred embodiment, the proton pump may be a microbial rhodopsin that is outwardly rectifying, and in particular may be derived from the halorubrum genus of archaeabacteria or leptosphaeria maculans, P. triticirepentis, and S. scelorotorium. The voltage potential of the target cell, subcellular region, or extracellular region may be increased or decreased until it is hyperpolarized, particularly to achieve neural silencing. A plurality of light-activated proton pumps responsive to different wavelengths of light may be used to achieve multi-color neural silencing.

In another aspect, the present invention is a method for adjusting the pH of cells, subcellular regions, or extracellular regions, comprising incorporating at least one gene encoding for a light-driven proton pump into at least one target cell, subcellular region, or extracellular region, the proton pump operating to change cell, subcellular region, or extracellular region pH in response to a specific wavelength of light, and causing the expression of the gene by exposing the target cell, subcellular region, or extracellular region to the specific wavelength of light in a manner designed to cause the pH of the target cell, subcellular region, or extracellular region to increase or decrease. In a preferred embodiment, the proton pump may be a microbial rhodopsin that is outwardly rectifying, and in particular may be derived from the halorubrum genus of archaeabacteria or leptosphaeria maculans, P. triticirepentis, and S. scelorotorium.

In yet another aspect, the present invention is a method for causing cells, subcellular regions, or extracellular regions to release protons as chemical transmitters, comprising incorporating at least one gene encoding for a light-driven proton pump into at least one target cell, subcellular region, or extracellular region, the proton pump operating to cause proton release in response to a specific wavelength of light, and causing the expression of the gene by exposing the target cell, subcellular region, or extracellular region to the specific wavelength of light in a manner designed to cause the target cell, subcellular region, or extracellular region to release protons. In a preferred embodiment, the proton pump may be a microbial rhodopsin that is outwardly rectifying, and in particular may be derived from the halorubrum genus of archaeabacteria or leptosphaeria maculans, P. triticirepentis, and S. scelorotorium.

### BRIEF DESCRIPTION OF THE DRAWINGS

Other aspects, advantages and novel features of the invention will become more apparent from the following detailed description of the invention when considered in conjunction with the accompanying drawings wherein:

FIG. 1 is a comparison graph depicting optical neural silencing by light-driven proton pumping, as revealed by a cross-kingdom functional molecular screen;

FIGS. 2A-I present functional properties of the light-driven proton pump Arch in neurons. In particular, FIGS. 2A and 2B are graphs of the photocurrents of Arch versus Halo measured as a function of light irradiance in patch-clamped cultured neurons for low and high light powers, respectively; FIG. 2C is an action spectrum of Arch measured in cultured neurons by scanning illumination light wavelength through the visible spectrum; FIG. 2D is a histogram depicting the photocurrent of Arch measured as a function of ionic composition; FIG. 2E is a plot of Arch proton photocurrent vs. holding potential; FIG. 2F is a histogram of Trypan blue staining of neurons lentivirally-infected with Arch vs. wild-type neurons; FIGS. 2G-I are histograms of membrane capacitance, membrane resistance, and resting potential in neurons lentivirally-infected with Arch vs. wild-type neurons, respectively;

FIGS. 3A-C graphically depict multicolor silencing of two neural populations, enabled by blue- and red-light drivable ion pumps of different classes. In particular, FIG. 3A is a graph depicting the action spectra of Mac versus Halo; FIG. 3B is FIG. 3B is a graph depicting membrane hyperpolarizations elicited by blue versus red light, in cells expressing Halo or Mac; and FIG. 3C graphically depicts action potentials evoked by current injection into patch-clamped cultured neurons transfected with Halo and Mac, that are selectively silenced with red vs. blue light;

FIGS. 4A-G depict results from an experimental implementation of the invention, using high-performance Arch-mediated optical neural silencing of neocortical regions in awake mice. In particular, FIGS. 4A and 4B are fluorescence images showing Arch-GFP expression in the mouse cortex, 1 month after lentiviral injection; FIG. 4C presents four representative extracellular recordings showing neurons undergoing 5-s, 15-s and 1-min periods of light illumination; FIG. 4D presents a spike raster plot of neural activity in a representative neuron before, during and after 5s of yellow light illumination, is shown as a spike raster plot, and a histogram of instantaneous firing rate averaged across trials; FIG. 4E presents in vitro data showing, in cultured neurons expressing Arch or eNpHR and receiving trains of somatic current, the percent reduction of spiking under varying light; FIG. 4F is a histogram depicting average change in spike firing during 5 seconds of yellow light illumination and during the 5 seconds immediately after light offset, for the data shown in FIG. 4D; and FIG. 4G is a histogram of percentage reductions in spike rate;

FIGS. 5A and 5B-D depict the temporally precise, reversible, repeatable, and cell type-specific silencing of the non-human primate via Arch and ArchT, respectively;

FIG. 6 is a graph demonstrating that Arch recovers spontaneously in the dark to its original state after prolonged illumination;

FIG. 7 is a graph of intracellular pH measurements in neurons expressing Arch over a 1-min period of continuous illumination and simultaneous imaging, depicting the use of proton pumps to alter intracellular pH using light according to one aspect of the present invention;

FIG. 8 is a graph showing peak current density recorded from wild type leptosphaeria maculans rhodopsin and various mutants;

FIG. 9 is a raw current trace recorded in a HEK cell expressing ArchT(w), illuminated with orange light followed by near-ultraviolet light, demonstrating bi-directional control of membrane voltage using two different colors of light to address one molecule;

FIG. 10 is a plot depicting bi-directional optical control of an archaerhodopsin “W96Y-like” variant derived from Halorubrum strain aus-2, according to one aspect of the present invention;

FIG. 11 is a plot depicting optically induced shunt-like activity exhibited by an archaerhodopsin “W96Y-like” variant derived from Halorubrum strain aus-1, according to one aspect of the present invention;

FIG. 12A is a histogram of photocurrents measured in cultured neurons expressing all known electrogenic archaerhodopsin full sequence clones, demonstrating that the class of proton pumps from halorubrum perform exceptionally well under mammalian physiological conditions;

FIG. 12B is a confocal fluorescence image of cultured neuron expressing Arch with a GFP fused to the C-terminus, showing good membrane localization in the absence of the appended signal sequences;

FIG. 13 is a histogram depicting the cumulative effect of appending signal sequences to a naturally occurring protein sequence;

FIGS. 14A and 14B are fluorescence images showing HEK293 cells transfected with MTS8-GFP and MTS8-ArchT-GFP plasmids, respectively; and

FIG. 15 is a trace of the S. sclerotorium opsin in an HEK293 cell.

### DETAILED DESCRIPTION

In the present invention, the expression, in genetically-targeted cells, of certain classes of genes encoding for light-driven proton pumps enables powerful hyperpolarization of cellular voltage in response to pulses of light. These pumps can be genetically-expressed in specific cells (for example, but not limited to, by using a virus) and then used to control cells in intact organisms (including, but not limited to, humans), as well as cells in vitro, in response to pulses of light. The magnitude of the current that can be pumped into cells expressing these pumps, upon exposure to light, is up to 16× greater than that of state-of-the-art pumps (e.g., Halo/NpHR [Han, X. & Boyden, E. S. (2007) PLoS ONE 2, e299; Zhang, F., Wang, L. P., Brauner, M., Liewald, J. F., Kay, K., Watzke, N., Wood, P. G., Bamberg, E., Nagel, G., Gottschalk, A., et al. (2007) Nature 446, 633-639]). Because the pumps of the present invention have different activation spectra from one another and from the state of the art natural gene products (e.g., Halo/NpHR), they also enable multiple colors of light to be used to hyperpolarize different sets of cells in the same tissue by expressing pumps with different activation spectra genetically in different cells and then illuminating the tissue with different colors of light.

In particular, the present invention employs two classes of light-activated proton pumps to hyperpolarize excitable cells: Microbial rhodopsins, such as the Halorubrum sodomense gene for archaerhodopsin-3 (herein abbreviated “Arch”) and Halorubrum strain TP009 gene for archaerhodopsin-TP009 (herein abbreviated “ArchT”), and eukaryotic proton pumps, such as leptosphaeria maculans (herein abbreviated “Mac”), P. triticirepentis, and S. sclerotorium rhodopsins. These proton pumps can also be used to modify the pH of cells and/or to release protons as chemical transmitters.

As used herein, the following terms expressly include, but are not to be limited to:

“Proton pump” means an integral membrane protein that is capable of moving protons across the membrane of a cell, mitochondrion, or other subcellular compartment.

Most proton pumps do not express well in mammalian cells [Chow, B. Y., Han, X., Dobry, A. S., Qian, X., Chuong, A. S., Li, M., Henninger, M. A., Belfort, G. M., Lin, Y., Monahan, P. E., et al. (2010) Nature 463, 98-102]. It was therefore necessary to screen a great many proton pumps in order to identify the class of microbial archaerhodopsins that was determined in the present invention to function better in mammalian cells than did other classes of proton pumps. The present invention therefore includes the discovery that microbial rhodopsins can be used in mammalian cells without need for any kind of chemical supplement, and in normal cellular environmental conditions and ionic concentrations. Specifically, it was shown that archaerhodopsins (proton pumps from Halorubrum) [Mukohata, Y., Ihara, K., Tamura, T., & Sugiyama, Y. (1999) J Biochem 125, 649-657], such as the Halorubrum sodomense gene for archaerhodopsin-3 (herein abbreviated “Arch”) [Chow, B. Y., Han, X., Dobry, A. S., Qian, X., Chuong, A. S., Li, M., Henninger, M. A., Belfort, G. M., Lin, Y., Monahan, P. E., et al. (2010) Nature 463, 98-102; Ihara, K., Umemura, T., Katagiri, I., Kitajima-Ihara, T., Sugiyama, Y., Kimura, Y., & Mukohata, Y. (1999) J Mol Biol 285, 163-174] and Halorubrum strain TP009 gene for archaerhodopsin-TP009 (herein abbreviated “ArchT”) [Sharma, A., Walsh, D. A., Bapteste, E., Rodriguez-Valera, F., Doolittle, W. F., Papke, R. T., (2007) BMC Evol Biol. 7, 79] encode for genes that, in humanized or mouse-optimized form, enable hyperpolarizations significantly larger than what has been discovered before.

Besides the microbial archaerhodopsins, a Leptosphaeria maculans rhodopsin [Waschuk, S. A., Bezerra, A. G., Shi, L., & Brown, L. S. (2005) Proceedings of the National Academy of Sciences of the United States of America 102, 6879-6883] also was found to work well, including double point mutants that were found to outperform all reported molecules to date. In particular, Leptosphaeria maculans rhodopsin responds strongly to blue light, and since other opsins identified to hyperpolarize cells respond to green, yellow, or reddish light, Leptosphaeria maculans rhodopsin can be expressed in a separate population of cells from a population of cells expressing one of these other opsins, thus allowing multiple colors of light to be used to silence these two populations of cells or neuronal projections from one site at different times [Chow, B. Y., Han, X., Dobry, A. S., Qian, X., Chuong, A. S., Li, M., Henninger, M. A., Belfort, G. M., Lin, Y., Monahan, P. E., et al. (2010) Nature 463, 98-102].

The present invention was reduced to practice in the laboratory by genetically expressing these molecules in excitable cells, illuminating the cells with light, and demonstrating rapid hyperpolarization of these cells in response to light, as well as rapid release from hyperpolarization upon cessation of light. The ability to controllably alter intracellular pH with light was also demonstrated, as was the ability to control membrane conductance bi-directional control via single molecule type that can depolarize or hyperpolarize a neuron with different colors of light or different light intensities. Thus, the present invention enables light-control of cellular functions in vivo (including in the non-human primate, which demonstrates pre-clinical enablement in humans) and in vitro, and accordingly has broad-ranging impact on prosthetics, drug screening, and other biotechnological areas, as discussed below.

In order to identify the opsins of the present invention, type I microbial opsins from archaebacteria, bacteria, plants, and fungi were screened for light-driven hyperpolarizing capability. Table 1 lists the modular screening candidates, including abbreviations, molecule classification, species of origin, and GenBank accession number (non-codon optimized). In Table 1, major molecule types are defined as “bacteriorhodopsins” or “rhodopsins” for outwardly rectifying proton pumps and “halorhodopsins” for inwardly rectifying chloride pumps. Sub-classifications of molecule type are determined by kingdom and/or genus of species of origin (e.g. archaerhodopsin=proton pump from halorubrum genus of halobacteria/haloarchaea).

TABLE 1

| | | Species of | GENBANK |
|---|---|---|---|
| Abbreviations | Molecule class | origin | Accession |
| Halo, NpHR, pHR | halorhodopsin | Natronomas | ABQ08589 |
| | | pharaonis | |
| sHR, HR | halorhodopsin | Halobacterium | NP_279315 |
| | | salinarum | |
| aHR-3 | archaehalorhodopsin | Halorubrum | BAA75202 |
| | | sodomense | |
| aHR-1, SGHR | archaehalorhodopsin | Halorubrum | CAA49773 |
| | | aus-1 (sp. SG1) | |
| cHR-3 | cruxhalorhodopsin | Haloarcula | BAA06679 |
| | | vallismortis | |
| cHR-5 | cruxhalorhodopsin | Haloarcula | AAV46572 |
| | | marismortui | |
| SquareHOP | square halorhodopsin | Haloquadratum | CAJ53165 |
| | | walysbyi | |
| dHR-1 | deltahalorhodopsin | Haloterrigena sp. | BAA75201 |
| | | Arg-4 | |
| SalHO, | bacterial halorhodopsin | Salinibacter ruber | AAT76430 |
| SRU_2780 | | | |
| pop | fungal opsin related | Podospora | XP_001904282 |
| | protein | anserina | |
| | | (DSM980) | |
| nop-1 | fungal opsin related | Neospora crassa | XP_959421 |
| | protein | | |
| Mac, LR, Ops | Fungal opsin related | Leptosphaeria | AAG01180 |
| | protein, | maculans | |
| | bacteriorhodopsin | | |
| Arch, aR-3 | archaerhodopsin | Halorubrum | BAA09452 |
| | | sodomense | |
| BR | Bacteriorhodopsin | Halobacterium | CAA23744 |
| | | salinarum | |
| cR-1 | Cruxrhodopsin | Haloarcula | BAA06678 |
| | | argentinensis | |
| | | (sp. arg-1 | |
| gPR, BAC31A8 | Proteorhodopsin | γ- | AAG10475 |
| | | proteobacterium | |
| | | BAC31A8 | |
| bPR, Hot75m4 | proteorhodopsin | γ- | Q9AFF7 |
| | | proteobacterium | |
| | | Hot75m4 | |
| Ace, AR | Algal bacteriorhodopsin | Acetabularia | AAY82897 |
| | | acetabulum | |

Mammalian codon-optimized genes were synthesized, cloned into GFP-fusion expression vectors, and transfected into cultured neurons. Opsin photocurrents and cell capacitance-normalized photocurrent densities were then measured under stereotyped illumination conditions, as well as opsin action spectra (photocurrent as a function of wavelength). The action spectra of screened candidates are presented in Table 2. In Table 2, reported peaks and full-width at half-maximum values are from second order Gaussian fits, in order to account for the characteristic “shoulder” of rhodopsins. “Spectral Screen Normalization Factor” accounts for differences in measured photocurrents due to varying excitation efficiencies via use of limited bandpass illumination filters (575±25 nm, 535±25 nm) for all molecules in the screen. All data reported was measured in neurons, except for Ace (Acetabularia acetabulum bacteriorhodopsin homolog), which was measured in HEK 293FT cells, in order to obtain a precise spectrum given the very low currents observed in neurons.

TABLE 2

| | | | | | Spectral Screen |
|---|---|---|---|---|---|
| | | | | | Normalization |
| | | | | Primary Peak ± | Factor, relative |
| | | | | FWHM (nm), | to Halo (see |
| | | Species of | GENBANK | second order | Experimental |
| Abbreviations | Molecule class | origin | Accession | Gaussian fit | Procedures) |
| Halo, NpHR, | halorhodopsin | Natronomas | ABQ08589 | 586 ± 52 | 1 |
| pHR | | pharaonis | | | |
| sHR, HR | halorhodopsin | Halobacterium | NP_279315 | No measured | N/A |
| | | salinarum | | photocurrent | |
| aHR-3 | archaehalorhodopsin | Halorubrum | BAA75202 | 586 ± 63 | 1.18 |
| | | sodomense | | | |
| aHR-1, SGHR | archaehalorhodopsin | Halorubrum | CAA49773 | No measured | N/A |
| | | aus-1 (sp. SG1) | | photocurrent | |
| cHR-3 | cruxhalorhodopsin | Haloarcula | BAA06679 | 592 ± 58 | 1.17 |
| | | vallismortis | | | |
| cHR-5 | cruxhalorhodopsin | Haloarcula | AAV46572 | 594 ± 52 | 1.10 |
| | | marismortui | | | |
| SquareHOP | square halorhodopsin | Haloquadratum | CAJ53165 | ¹⁴⁴³²⁷¹⁹¹⁹No | N/A |
| | | walysbyi | | measured | |
| | | | | photocurrent | |
| dHR-1 | deltahalorhodopsin | Haloterrigena | BAA75201 | No measured | N/A |
| | | sp. Arg-4 | | photocurrent | |
| SalHO, | bacterial | Salinibacter | AAT76430 | 582 ± 71 | 1.12 |
| SRU_2780 | halorhodopsin | ruber | | | |
| Pop | fungal opsin related | Podospora | XP_001904282 | No measured | N/A |
| | protein | anserina | | photocurrent | |
| | | (DSM980) | | | |
| nop-1 | fungal opsin related | Neospora | XP_959421 | No measured | N/A |
| | protein | crassa | | photocurrent | |
| Mac, LR, Ops | fungal opsin related | Leptosphaeria | AAG01180 | 550 ± 69 | 0.94 |
| | protein, | maculans | | | |
| | bacteriorhodopsin | | | | |
| Arch, aR-3 | archaerhodopsin | Halorubrum | BAA09452 | 566 ± 66 | 1.08 |
| | | sodomense | | | |
| BR | bacteriorhodopsin | Halobacterium | CAA23744 | 572 ± 75 | 1.26 |
| | | salinarum | | | |
| cR-1 | cruxrhodopsin | Haloarcula | BAA06678 | 557 ± 67 | 1.23 |
| | | argentinensis | | | |
| gPR, | proteorhodopsin | γ- | AAG10475 | No measured | N/A |
| BAC31A8 | | proteobacterium | | photocurrent | |
| | | BAC31A8 | | | |
| bPR, Hot75m4 | proteorhodopsin | γ- | Q9AFF7 | No measured | N/A |
| | | proteobacterium | | photocurrent | |
| | | Hot75m4 | | | |
| Ace, AR | algal | Acetabularia | AAY82897 | 505 ± 57 | 0.98 |
| | bacteriorhodopsin | acetabulum | | | |

From this information, the photocurrent density for each opsin was estimated at its own spectral peak. For comparison purposes, an earlier-characterized microbial opsin was included, the Natronomonas pharaonis halorhodopsin (Halo/NpHR), a light-driven inward chloride pump capable of modest hyperpolarizing currents. Archaerhodopsin-3 from Halorubrum sodomense (Arch/aR-3), hypothesized to be a proton pump, generated large photocurrents in the screen, as did two other proton pumps, the Leptosphaeria maculans opsin (Mac/LR/Ops) and cruxrhodopsin-1 (albeit less than that of Arch). All light-driven chloride pumps assessed had lower screen photocurrents than these light-driven proton pumps.

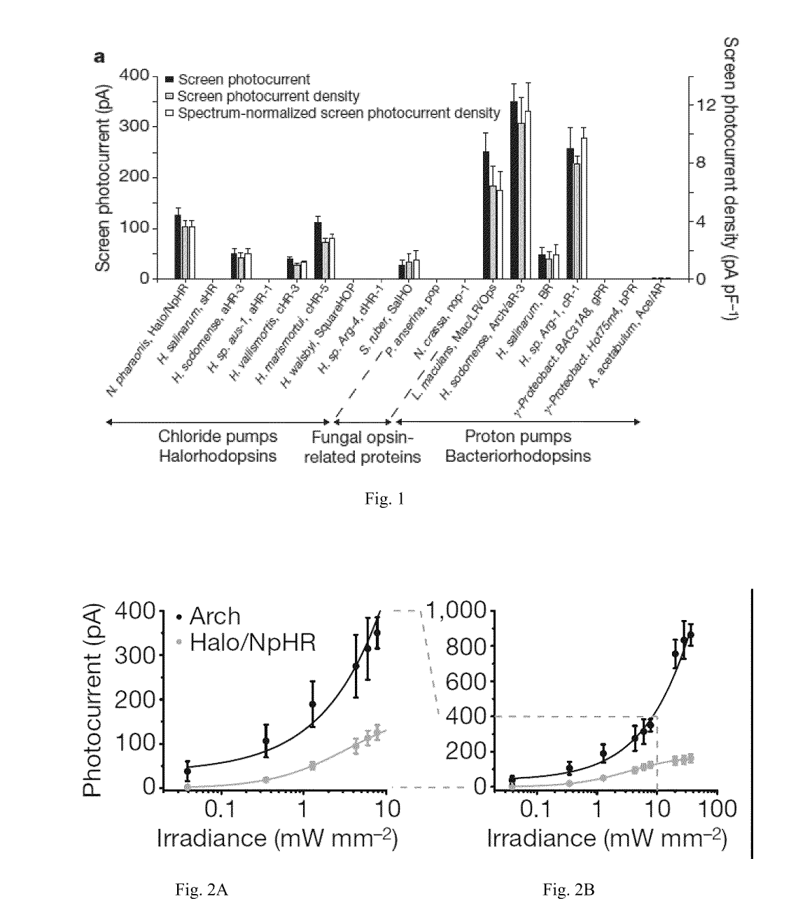

FIG. 1 is a comparison graph depicting optical neural silencing by light-driven proton pumping, as revealed by a cross-kingdom functional molecular screen. Shown in FIG. 1 are screen data showing outward photocurrents (left y axis, black bars), photocurrent densities (right y axis, grey bars), and action spectrum-normalized photocurrent densities (right y axis, white bars), measured by whole-cell patch-clamp of cultured neurons under screening illumination conditions (575±25 nm, 7.8 mW/mm² for all except Mac/LR/Ops, gPR, bPR and Ace/AR, which were 535±25 nm, 9.4 mWmm²; n=4-16 neurons for each bar). Data are mean+/−standard error. Full species names from left to right: Natronomonas pharaonis, Halobacterium salinarum, Halorubrum sodomense, Halorubrum species aus-1, Haloarcula vallismortis, Haloarcula marismortui, Haloquadratum walsbyi, Haloterrigena species Arg-4, Salinibacter rubes; Podospora anserina, Neurospora crassa, Leptosphaeria maculans, Halorubrum sodomense, Halobacterium salinarum, Haloarcula species Arg-1, uncultured gamma-proteobacterium BAC31A8, uncultured gamma-proteobacterium Hot75m4 and Acetabularia acetabulum.

Compared to the currently reported natural gene sequences used to silence neurons in the prior art [Halo/NpHR [Han, X. & Boyden, E. S. (2007) PLoS ONE 2, e299; Zhang, F., Wang, L. P., Brauner, M., Liewald, J. F., Kay, K., Watzke, N., Wood, P. G., Bamberg, E., Nagel, G., Gottschalk, A., et al. (2007) Nature 446, 633-639], Arch and ArchT have demonstrably improved photocurrent generation at all illumination wavelengths. Arch is a yellow-green light sensitive opsin that appears to express well on the neural plasma membrane. Arch-mediated currents exhibited excellent kinetics of light-activation and post-light recovery. Upon illumination, Arch currents rose with a 15%-85% onset time of 8.8±1.8 ms (mean±standard error (SE) reported throughout, unless otherwise indicated; N=16 neurons), and after light cessation, Arch currents fell with an 85%-15% offset time of 19.3±2.9 ms. Under continuous yellow illumination, Arch photocurrent declined, as did the photocurrents of all of the opsins in the screen. However, Arch spontaneously recovered function in seconds, more like the light-gated cation channel channelrhodopsin-2 (ChR2).

The observed behavior of Arch is unlike all of the other halorhodopsins screened, including products of halorhodopsin site-directed mutagenesis aimed at improving kinetics, which after illumination remained inactivated for long periods of time (e.g., tens of minutes, with accelerated recovery requiring additional blue light). Table 3 presents action spectrum and spontaneous recovery to active pumping state in the dark for N. pharaonis halorhodopsin (Halo, NpHR) point mutants examined in HEK293FT cells. In Table 3, reported peaks and full-width at half-maximum values are from second order Gaussian fits, in order to account for the characteristic “shoulder” of rhodopsins. The column “Primary predicted outcome of mutation” lists hypothesized outcomes as to what parameters of molecular performance might be expected to change, for each mutation. The term “Recovery” refers to spontaneous recovery of the active pumping state in the dark over a timescale of seconds, which holds for Arch and ChR2 but not for Halo. “Recovery” candidate residues were targeted based on their hypothesized roles in chloride affinities and/or transport kinetics, as determined by structure-function studies and mutation studies using other halorhodopsins. Spectral residues were targeted based on their predicted retinal flanking locations based on crystal structures, and/or have been shown previously to govern the spectrum of bacteriorhodopsin. Alignments to H. salinarum halorhodopsin and bacteriorhodopsins were made using NCBI Blast.

TABLE 3

| | | | Primary | Primary Peak ± | |
|---|---|---|---|---|---|
| | Homologous | Homologous | predicted | FWHM (nm), | Recovery of |
| Halo point | H. salinarum | H. salinarum | outcome of | second order | active pumping |
| mutant | HR residue | BR residue | mutation | Gaussian fit | in dark? |
| Wild type | N/A | N/A | N/A | 584 ± 51 | No |
| T126H | T111 | D85 | Recovery | No measured | No |
| | | | | photocurrent | |
| T126R | T111 | D85 | Recovery | No measured | No |
| | | | | photocurrent | |
| W127F | W112 | W86 | Spectral shift | No measured | No |
| | | | | photocurrent | |
| S130T | S130 | T89 | Recovery | 568 ± 55 | No |
| S130D | S130 | T89 | Recovery | No measured | No |
| | | | | photocurrent | |
| S130H | S130 | T89 | Recovery | No measured | No |
| | | | | photocurrent | |
| S130R | S130 | T89 | Recovery | No measured | No |
| | | | | photocurrent | |
| A137T | A122 | D96 | Recovery | 585 ± 52 | No |
| A137D | A122 | D96 | Recovery | 575 ± 53 | No |
| A137H | A122 | D96 | Recovery | No measured | No |
| | | | | photocurrent | |
| A137R | A122 | D96 | Recovery | No measured | No |
| | | | | photocurrent | |
| G163C | G148 | G122 | Spectral shift | No measured | No |
| | | | | photocurrent | |
| W179F | W164 | W137 | Spectral shift | No measured | No |
| | | | | photocurrent | |
| S183C | F168 | S141 | Spectral shift | No measured | No |
| | | | | photocurrent | |
| F187M | F172 | M145 | Spectral shift | 589 ± 52 | No |
| F187A | F172 | M145 | Spectral shift | No measured | No |
| | | | | photocurrent | |
| K215H | R200 | R149 | Recovery | 586 ± 50 | No |
| K215R | R200 | R149 | Recovery | 575 ± 51 | No |
| K215Q | R200 | R149 | Recovery | 585 ± 56 | No |
| T218S | T203 | T178 | Recovery | 582 ± 53 | No |
| T218D | T203 | T178 | Recovery | No measured | No |
| | | | | photocurrent | |
| T218H | T203 | T178 | Recovery | No measured | No |
| | | | | photocurrent | |
| T218R | T203 | T178 | Recovery | No measured | No |
| | | | | photocurrent | |
| W222F | W207 | W182 | Spectral shift | No measured | No |
| | | | | photocurrent | |
| P226V | P211 | P186 | Spectral shift | No measured | No |
| | | | | photocurrent | |
| P226G | P211 | P186 | Spectral shift | No measured | No |
| | | | | photocurrent | |
| W229F | W214 | W189 | Spectral shift | 587 ± 53 | No |

Describing the methods employed in brief: codon-optimized genes were synthesized by Genscript and fused to GFP in lentiviral and mammalian expression vectors as used previously for transfection or viral infection of neurons. Primary hippocampal or cortical neurons were cultured and then transfected with plasmids or infected with viruses encoding for genes of interest, as described previously⁵. Images were taken using a Zeiss LSM 510 confocal microscope. Patch clamp recordings were made using glass microelectrodes and a Multiclamp 700B/Digidata electrophysiology setup, using appropriate pipette and bath solutions for the experimental goal at hand. Neural pH imaging was done using carboxy-SNARF-1-AM ester (Invitrogen). Cell health was assayed using Trypan blue staining (Gibco). HEK cells were cultured and patch clamped using standard protocols. Mutagenesis was performed using the QuikChange kit (Stratagene). Computational modelling of light propagation was done with Monte Carlo simulation with MATLAB. In vivo recordings were made on headfixed awake mice, which were surgically injected with lentivirus, and implanted with a headplate as described before. Glass pipettes attached to laser-coupled optical fibers were inserted into the brain, to record neural activity during laser illumination in a photoelectrochemical artifact-free way. Data analysis was performed using Clampfit, Excel, Origin, and MATLAB. Histology was performed using transcardial formaldehyde perfusion followed by sectioning and subsequent confocal imaging.

Outline of the method used for quantitative analysis of opsin-GFP membrane expression in neurons, modified from Wang, H. et al. Molecular determinants differentiating photocurrent properties of two channelrhodopsins from chlamydomonas. J Biol Chem 284, 5685-5696 (2009). The cell contour was first enhanced using the blur and subtraction methodology as described in step C of Supplementary Figure S2 of Wang et al. The Magic Wand tool in ImageJ was used to define the pixels corresponding to the cell membrane. However, the tool sometimes selected the whole somatic cytoplasm and the processes, because some neuronal processes were too small to be separated into membrane vs. cytosol, causing the appearance of connectedness, and/or because the well-defined membrane processes overlap with other neurons or extend to the edge of the image. Line sections were generated at the apparent boundary of the soma and its processes, to separate sub-resolution image components from the soma (drawn as red here). The Magic Wand tool could now select distinct membrane segments of the soma. Membrane expression was then quantified by taking the area-weighted average of membrane pixel values, in the original image.

Using this method, it was found that the absolute expression level of Arch on the plasma membrane was similar to that of Halo (p>0.2, N=5 cells each). Experimenting with adding targeting sequences that improve trafficking showed that adding a signal sequence from the MHC Class I antigen (‘ss’) as well as the Kir2.1 ER export sequence (‘ER2’) (which is part of the sequence that boosts Halo currents by 75%, resulting in eNpHR [Gradinaru, V., Thompson, K. R. Deisseroth, K., (2008) Brain Cell, 129-139]), did not augment Arch currents (p>0.6, N=16 Arch cells, N=9 ss-Arch-ER2 cells). Thus, the effect of a given trafficking-improving sequence on opsin expression is opsin-specific (and perhaps species-specific), but nonetheless deserves further attention. For example, adding the Prolactin signal sequence (‘Prl’) to the N terminus of Arch trended towards boosting the Arch current (+32%; p<0.08, N=18 ss-Prl-Arch cells). Improving opsin targeting, however, is unlikely to alter opsin recovery kinetics or light dynamic range.

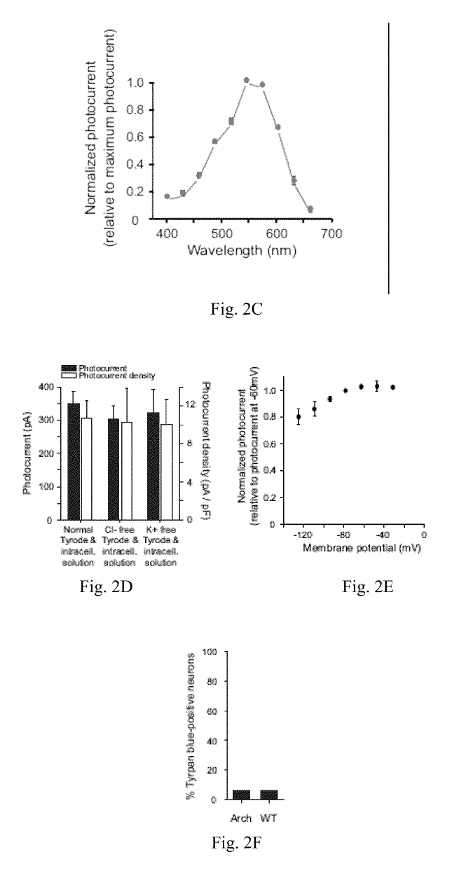

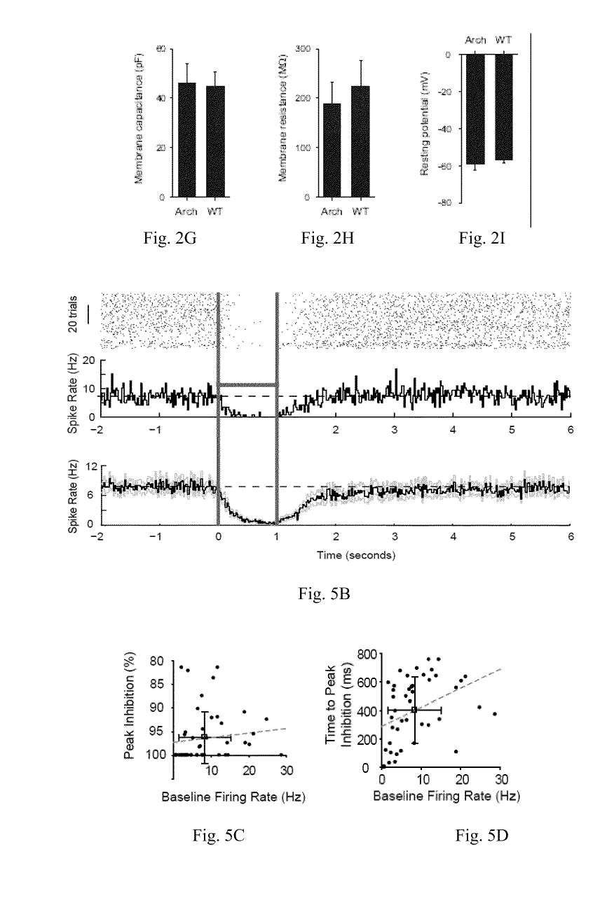

FIGS. 2A-I present functional properties of the light-driven proton pump Arch in neurons. FIGS. 2A and 2B are graphs of the photocurrents of Arch versus Halo measured as a function of 575±25 nm light irradiance in patch-clamped cultured neurons (n=4-16 neurons for each point), for low (FIG. 2A) and high (FIG. 2B) light powers. As seen in FIGS. 2A and 2B, Arch produces between a 2.7× and 16× more photocurrent than the current state-of-the-art gene product (N. pharaonis halorhodopsin, also denoted as “NpHR” or “Halo”) at 6 mW/mm² yellow light illumination and 0.04 mW/mm², respectively (wavelength=575±25 nm). FIG. 2C is an action spectrum of Arch measured in cultured neurons by scanning illumination light wavelength through the visible spectrum (N=7 neurons).

FIG. 2D depicts the photocurrent of Arch measured as a function of ionic composition (575±25 nm light, 7.8 mW/mm²), showing no significant dependence of photocurrent on concentration of Cl− or K+ ions (N=16, 8 and 7 neurons, from left to right). FIG. 2E depicts Arch proton photocurrent vs. holding potential (N=4 neurons). Together, FIGS. 2D and 2E demonstrate conclusively that Arch functions as a proton pump.

FIG. 2E is a histogram of Trypan blue staining of neurons lentivirally-infected with Arch vs. wild-type (WT) neurons, measured at 18 days in vitro (N=669 Arch-expressing, 512 wild-type, neurons). FIGS. 2G-I are histograms of membrane capacitance (FIG. 2G), membrane resistance (FIG. 2H), and resting potential (FIG. 2I) in neurons lentivirally-infected with Arch vs. wild-type (WT) neurons, measured at 11 days in vitro (N=7 cells each). Together, FIGS. 2F-I demonstrate that expression of Arch does not harm or alter the physiology of the neurons in which it is expressed.

The magnitude of Arch-mediated photocurrents was large. At low light irradiances of 0.35 and 1.28 mW/mm², neural Arch currents were 120 and 189 pA respectively; at higher light powers (e.g., at which Halo currents saturate), Arch currents continued to increase, approaching 900 pA at effective irradiances of 36 mW/mm², well within the reach of typical in vivo experiments. The high dynamic range of Arch may enable excellent utilization of light sources (e.g., LEDs, lasers) that are safe and effective for optical control in vivo.

Several lines of evidence supported the idea that Arch functioned as an outward proton pump when expressed in neurons. Removing the endogenous ions that commonly subserve neural inhibition, Cl⁻ and K⁺, from physiological solutions did not alter photocurrent magnitude (p>0.4 comparing either K⁺ free or Cl⁻ free solutions to regular solutions, t-test). In solutions lacking Na⁺, K⁺, Cl⁻, and Ca²⁺, photocurrents were still no different from those measured in normal solutions (p>0.4; N=4 neurons tested without these four charge carriers). The reversal potential appeared to be less than −120 mV, also consistent with Arch being a proton pump.

The voltage swings driven by illumination of current-clamped Arch-expressing cultured neurons were assessed. As effective irradiance increased from 7.8 mW/mm² to 36.3 mW/mm², voltage clamped neurons exhibited peak currents that increased from 350±35 pA (N=16 neurons) to 863±62 pA (N=8 neurons) respectively. Current-clamped neurons under these two irradiance conditions were hyperpolarized by −69.6±7.3 mV (N=10) and −76.2±10.1 mV (N=8) respectively. Surprisingly, these voltage deflections, while both large, were not significantly different from one another (p>0.7, t-test), suggesting the existence of a rapidly activated transporter or exchanger (perhaps the Na⁺-dependent Cl⁻/HCO₃⁻ exchanger), or the opening of hyperpolarization-gated channels capable of shunting protons, which limit the effects of Arch on accumulated proton (or other charge carrier) gradients across neural membranes. This enabling of effective but not excessive silencing may make Arch safer than pumps that accumulate ions without self-regulation.

The changes in intracellular pH (pHᵢ) driven by illumination of Arch-expressing cultured neurons was assessed, using the fluorescent pH indicator carboxy-SNARF-1. Within one second of illumination with strong green light, pH, rose from 7.309±0.011 to 7.431±0.020, plateauing rapidly. pHᵢ increased slightly further after 15 of illumination, to 7.461±0.024. The fast stabilization of pHᵢ may reflect the same self-limiting influence that limits proton-mediated voltage swings as described above, and may contribute to the safe operation of Arch in neurons by preventing large pHᵢ swings. The changes in pH, observed here are comparable in magnitude to those observed during illumination of ChR2-expressing cells¹³ (due to the proton currents carried by ChR2^{3,14}) and are also within the magnitudes of changes observed during normal neural activityl⁵⁻¹⁸. Passive electrical properties of neurons were not affected by Arch expression (p>0.6 for each measure, t-test), nor was cell death (p>0.6, χ=0.26).

The tissue volumes that could be silenced were estimated, using in vitro experiments and computational modeling. In cultured neurons expressing Arch or a trafficking-improved variant of Halo, eNpHR, brief current pulses were somatically injected at magnitudes chosen to mimic the current drives of neurons in the intact nervous system. These neurons were exposed to periods of 575 nm yellow light (0.35, 1.28, or 6 mW/mm², simulating irradiance ˜1.7, 1.2, or 0.6 mm away from the tip of a 200 micron fiber emitting 200 mW/mm² irradiance, as modelled by Monte Carlo methods, and measured the reduction in spike rate for each condition. In general, Arch-expressing neurons were significantly more inhibited than eNpHR-expressing cells. According to the model and the 350 pA data, the increase in brain tissue volume that would be 45-55% optically silenced would be ˜10× larger for Arch than for eNpHR.

Proton pumps naturally exist that are activated by many colors of light, in contrast to chloride pumps, which are primarily driven by yellow-orange light (even with significant mutagenesis of retinal-flanking residues). One aspect of the invention is the use of the proton pumping rhodopsin genes from eukaryotes, such as algae, such as Acetabularia acetabulum, or fungus, such as Leptosphaeria maculans, P. tritici-repentis, and S. sclerotorium. These fungal rhodopsins have been identified as particularly efficacious light-activated proton pumps.

In particular, the present invention demonstrates that leptosphaeria maculans rhodopsin can hyperpolarize cells in strong response to blue light with sufficient spectral independence from the majority of electrogenic hyperpolarizing microbial rhodopsins that absorb more red-shifted light to enable the use of multiple colors of light to hyperpolarize different sets of cells in the same tissue by expressing pumps with different activation spectra genetically in different cells and then illuminating the tissue with different colors of light. For example, if one set of cells in a tissue (for example, but not limited to, excitatory neurons) express Halo, and a second set express Arch, then illuminating the tissue with 630 nm light will preferentially hyperpolarize the first set, whereas illuminating the tissue with 470 nm light will preferentially hyperpolarize the second set. Other pairs of targets that can be modulated with two colors of light in the same illumination area include, but are not limited to, two projections to/from one site, or combinations of the cell, its projections, and its organelles, given the ability to target the molecule sub-cellularly (Lewis et al. demonstrate that such localization is possible [Lewis, T. L., Jr., Mao, T., Svoboda, K., & Arnold, D. B. (2009) Nature neuroscience 12, 568-576]).

The light-driven proton pump Mac had an action spectrum strongly blueshifted relative to that of the light-driven chloride pump Halo. It was found that Mac-expressing neurons could undergo 4.1-fold larger hyperpolarizations with blue light than with red light, and Halo-expressing neurons could undergo 3.3-fold larger hyperpolarizations with red light than with blue light, when illuminated with appropriate filters. Accordingly, selective silencing of spike firing in Mac-expressing neurons in response to blue light and selective silencing of spike firing in Halo-expressing neurons in response to red light were demonstrated. Thus, the spectral diversity of proton pumps points the way towards independent multi-color silencing of separate neural populations. This result opens up novel kinds of experiment, in which, for example, two neuron classes, or two sets of neural projections from a single site, can be independently silenced during a behavioral task

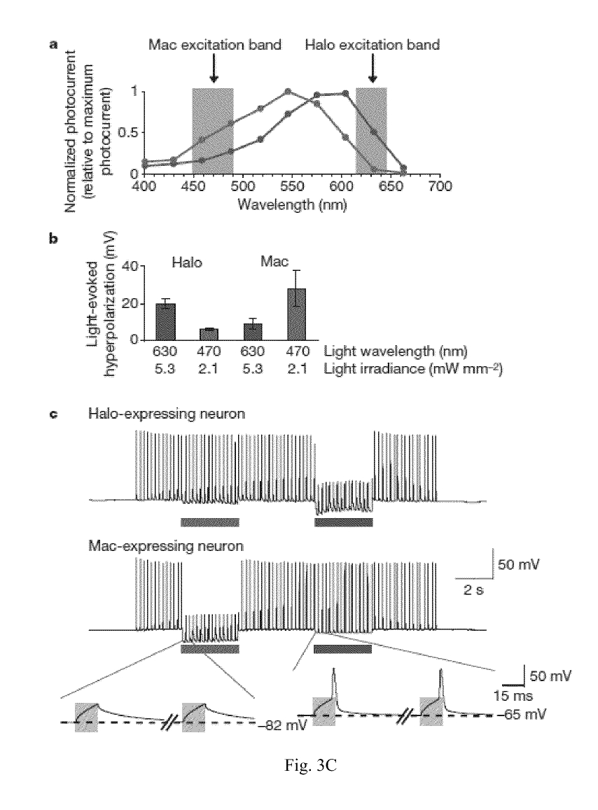

FIGS. 3A-C depict multicolor silencing of two neural populations, enabled by blue- and red-light drivable ion pumps of different classes. FIG. 3A is a graph depicting the action spectra of Mac versus Halo. In FIG. 3A, rectangles indicate filter bandwidths used for multicolour silencing in vitro. Blue light is delivered by a 470±20 nm filter at 5.3 mW/mm², and red light is delivered by a 630±15 nm filter at 2.1 mW/mm². FIG. 3B is a graph depicting membrane hyperpolarizations elicited by blue versus red light, in cells expressing Halo or Mac (n=5 Mac-expressing and n=6 Haloexpressing neurons). FIG. 3C graphically depicts that action potentials evoked by current injection into patch-clamped cultured neurons transfected with Halo (top) were selectively silenced by the red light but not by the blue light, and vice-versa in neurons expressing Mac (middle). Grey boxes in the inset (bottom) indicate periods of patch-clamp current injection.

In a preferred method for utilization of the invention, the following methodology is used. First, take the gene for either the Halobacterium sodomense gene for archaerhodopsin-3 (SEQ ID No. 1, Genbank accession # D50848.1), human codon-optimized DNA (SEQ ID No. 2, Genbank accession # GU045593, Genbank accession # GU045594 when fused to C-terminal GFP [Chow, B. Y., Han, X., Dobry, A. S., Qian, X., Chuong, A. S., Li, M., Henninger, M. A., Belfort, G. M., Lin, Y., Monahan, P. E., et al. (2010) Nature 463, 98-102]); or the gene for Halorubrum strain TP009 (SEQ ID No. 3, Genbank accession number ABT17417.1), mammalian codon-optimized DNA (SEQ ID No.4); or the gene for leptosphaeria maculans rhodopsin (SEQ ID No.5, Genbank accession # AAG01180.1), mammalian codon-optimized DNA (SEQ ID No.6, Genbank accession # GU045595, Genbank accession # GU045596 when fused to C-terminal GFP [Chow, B. Y., Han, X., Dobry, A. S., Qian, X., Chuong, A. S., Li, M., Henninger, M. A., Belfort, G. M., Lin, Y., Monahan, P. E., et al. (2010) Nature 463, 98-102; Waschuk, S. A., Bezerra, A. G., Shi, L., & Brown, L. S. (2005) Proceedings of the National Academy of Sciences of the United States of America 102, 6879-6883]), and express it in cells. In a preferred embodiment, the gene is expressed in cells according to the methodology that follows, an exemplary reduction to practice of which has been previously described by Chow et al. [Chow, B. Y., Han, X., Dobry, A. S., Qian, X., Chuong, A. S., Li, M., Henninger, M. A., Belfort, G. M., Lin, Y., Monahan, P. E., et al. (2010) Nature 463, 98-102].

To start, clone the opsin gene into a lentiviral or adeno-associated virus (AAV) packaging plasmid, or another desired expression plasmid, and then clone GFP downstream of the preferred gene, eliminating the stop codon of the opsin gene, thus creating a fusion protein. The viral or expression plasmid may contain either a strong general promoter, a cell-specific promoter, or a strong general promoter followed by one or more logical elements (such as a lox-stop-lox sequence, which will be removed by Cre recombinase selectively expressed in cells in a transgenic animal, or in a second virus, thus enabling the strong general promoter to then drive the gene [Atasoy, D., Aponte, Y., Su, H. H., & Steprnson, S. M. (2008) J Neurosci 28, 7025-7030; Kuhlman, S. J. & Huang, Z. J. (2008) PLoS ONE 3, e200]).

If using a viral plasmid, synthesize the viral vector using the viral plasmid, using standard techniques (e.g., Sena-Esteves, M., Tebbets, J. C., Steffens, S., Crombleholme, T., & Flake, A. W. (2004) J Virol Methods 122, 131-139). If using a virus, as appropriate for gene therapy (over 600 people have been treated with AAV carrying various genetic payloads to date, in 48 separate clinical trials, without a single adverse event), inject the virus using a small needle or cannula into the area of interest, thus delivering the gene encoding the opsin fusion protein into the cells of interest. If using another expression vector, directly electroporate or inject that vector into the cell or organism (for acutely expressing the opsin, or making a cell line, or a transgenic mouse or other animal).

Illuminate with light. For Arch, peak illumination wavelengths are 566 nm±66 nm (when incident intensity is defined in photons/second; second order Gaussian fit maximum±full width at half-maximum). For Mac, peak illumination wavelengths are 550 nm±69 nm. To illuminate two different populations of cells (e.g., in a single tissue) with two different colors of light, first target one population with Halo, and the other population with Mac, using two different viruses (e.g., with different coat proteins or promoters) or two different plasmids (e.g., with two different promoters). Then, after the molecule expresses, illuminate the tissue with 450±25 nm, 475±25 nm, or 500±25 nm light to preferentially hyperpolarize the Mac-expressing cells, and illuminate the tissue with 625±25 nm light, 600±25 nm light, or 575±25 nm light, to preferentially hyperpolarize the Halo-expressing cells. The above wavelengths illustrate typical modes of operation, but are not meant to constrain the protocols that can be used, and it will be clear to one of skill in the art of the invention that many other protocols are suitable for use in the present invention For example, either narrower or broader wavelengths, or differently-centered illumination spectra, can be used. For prosthetic uses, the devices used to deliver light may be implanted[Campagnola, L., Wang, H., & Zylka, M. J. (2008) J Neurosci Methods 169, 27-33]. For drug screening, a xenon lamp or LED can be used to deliver the light.

An experimental implementation of the invention demonstrated high-performance Arch-mediated optical neural silencing of neocortical regions in awake mice. Fluorescence images of a neuron in the awake mouse brain, expressing Arch, and silenced in response to yellow light, were recorded with a glass micropipette, thus demonstrating in vivo use of this molecule. Cells were healthy and tolerated the expression of the molecule well. After light illumination ceased, the neuron easily restored to its original, natural spike rate. This demonstrates the creation of temporary lesions of neural information content.

To directly assess Arch in vivo, lentivirus encoding for Arch was injected into mouse cortex and neural responses were recorded ˜1 month later. Arch expressed well and appeared well localized to the plasma membrane, labeling cell bodies, processes, and dendritic spines. Neurons in awake headfixed mice were recorded, illuminating neurons via a 200 micron optical fiber coupled to a 593 nm laser (power at electrode tip estimated at ˜3 mW/mm²). Upon light onset, firing rates of many units immediately and strongly declined, and remained low throughout the period of illumination, for both brief and long pulses. 13 single units were recorded that showed any decrease in firing during illumination, and spiking rates during exposure to 5s yellow light were found to drop by an average of 90±15% (mean±standard deviation (SD)), restoring to levels indistinguishable from baseline after light cessation (p>0.2, paired t-test). 6 of the 13 units decreased spike rate by at least 99.5%, and the median decrease was 97.1%. One possibility is that Arch-expressing cells were almost completely silenced, whereas non-infected cells decreased activity due to network activity reduction during illumination; note that only excitatory cells were genetically targeted here. Optical silencing was consistent across trials (p>0.1, paired t-test comparing, for each neuron, responses to first 3 vs. last 3 light exposures; ˜20 trials per neuron). The kinetics of silencing were rapid: for the 6 neurons that underwent >99.5% silencing, spike firing reduced with near-0 ms latency, rarely firing spikes after light onset; averaged across all cells, firing rate reductions plateaued within 229±310 ms after light onset (mean±SD). After light cessation, firing rate restored quickly for the highly-silenced neurons; averaged across all cells, firing rates took 355±505 ms to recover after light offset. The level of post-light firing did not vary with repeated light exposure (p>0.7, paired t-test comparing, for each neuron, after-light firing rates during first 3 vs. the last 3 trials). Thus, Arch could mediate reliable, near-digital silencing of neurons in the awake mammalian brain.

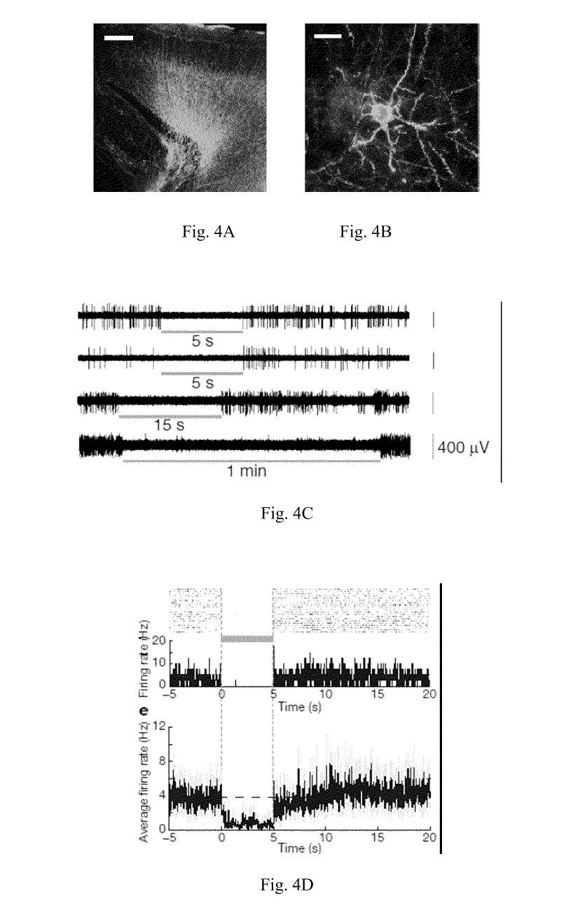

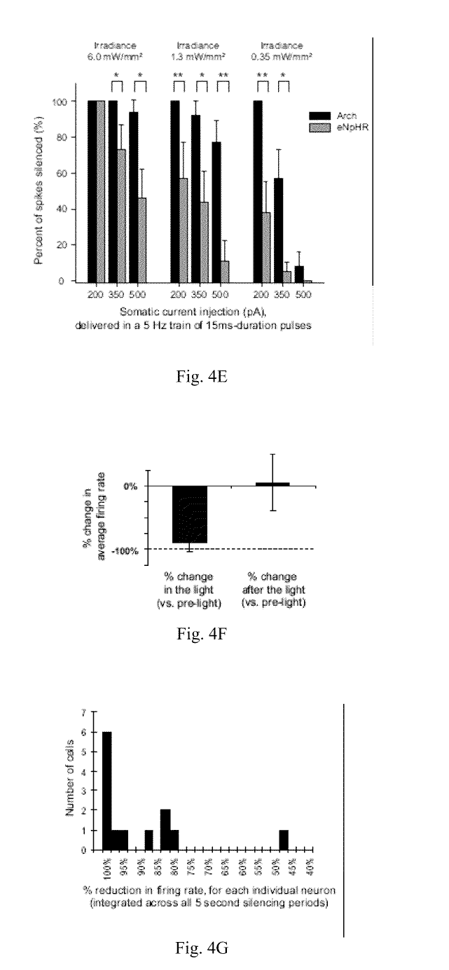

FIGS. 4A-G depict results from this experimental implementation of the invention, using high-performance Arch-mediated optical neural silencing of neocortical regions in awake mice. FIGS. 4A and 4B are fluorescence images showing Arch-GFP expression in the mouse cortex, 1 month after lentiviral injection. Scale bars, 200 um (left) and 20 um (right). FIG. 4C presents four representative extracellular recordings showing neurons undergoing 5-s, 15-s and 1-min periods of light illumination (593 nm; 150 mW/mm² radiant flux out the fibre tip; and expected to be 3 mW/mm² at the electrode tip, 800 mm away, based on Monte Carlos modeling). In FIG. 4D, neural activity in a representative neuron before, during, and after 5s of yellow light illumination, is shown as a spike raster plot and as a histogram of instantaneous firing rate averaged across trials (bottom; bin size, 20 ms). Population average of instantaneous firing rate before, during and after yellow light illumination (black line, mean; gray lines, mean±SE; n=13 units).

FIG. 4E presents in vitro data showing, in cultured neurons expressing Arch or eNpHR and receiving trains of somatic current injections (15 ms pulse durations at 5 Hz), the percent reduction of spiking under varying light powers (575±25 nm light) as might be encountered in vivo. *, p<0.05; **, p<0.01, t-test. N=7-8 cells for each condition, demonstrating that Arch performs better with statistical significance when compared to the state of the art. FIG. 4F is a histogram depicting average change in spike firing during 5 seconds of yellow light illumination (left) and during the 5 seconds immediately after light offset (right), for the data shown in FIG. 4D, demonstrating that illumination and silencing does not alter the excitability of the neuron. FIG. 4G is a histogram of percentage reductions in spike rate, demonstrating the high success rate using the present invention.

Another aspect of the invention is the reduction to practice of temporally precise, reversible, safe, and cell type-specific silencing of the awake primate brain, here shown in the macaque parietal cortex, silenced by Arch under 532 nm illumination. Such silencing has been demonstrated with both Arch and ArchT.

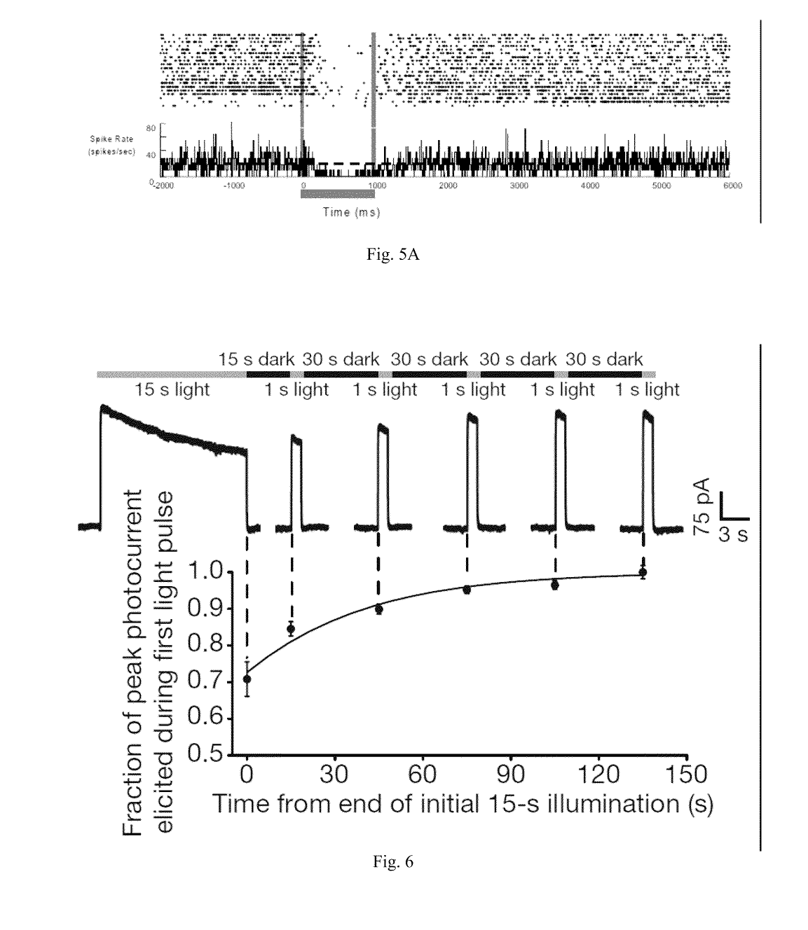

FIG. 5A depicts the temporally precise, reversible, repeatable, and cell type-specific silencing of the non-human primate via Arch (here, shown for the macaque parietal cortex). As seen in FIG. 5A, firing activity of a multi-unit, recorded in the Macaque parietal cortex was dramatically reduced upon exposure to 1 second green light (532 nm laser, blue dash) 2 months after injection of high titer lentivirus carrying Arch-GFP gene behind the 1.3 kb CaMKII promoter. (Top) Spike raster shows 20 trials. (Bottom) Histogram of instantaneous firing rate across all trials, bin=5 ms.

FIG. 5B depicts the temporally precise, reversible, repeatable, and cell type-specific silencing of the non-human primate via ArchT (here, shown for the macaque parietal cortex). As seen in FIG. 5B, upon green light illumination (532 nm), primate cortical neurons expressing ArchT decrease their firing rate (sample individual neuron, top) and achieved near 100% silencing after a few hundred milliseconds. On average, of the 46 single units, neurons reached peak silencing of 96.3%±5.5%, mean±standard deviation, n=46 neurons) 402±233 ms after light onset. 24 of the 46 neurons were 100% silenced (population average, middle). The amount of silencing achieved upon illuminating ArchT expressing neurons is independent of their baseline firing rates (linear regression, p=0.3, R̂2=0.022) (FIG. 5C). However, it trends to take longer to silence a neuron with higher baseline firing rate (linear regression p=0.007, R̂2=0.153) (FIG. 5D).

This demonstrates pre-clinical translational viability and technology viability, the latter as evident by the ability to silence large volumes of brain tissue (estimated to be larger than the primary volume of tissue that the virus infects) with far more numerous and active depolarizing synaptic inputs to overcome than in the anaesthetized or awake rodent. Unlike with electrical [Kern, D. S. & Kumar, R. (2007) Neurologist 13, 237-252] and electromagnetic stimulation [Kobayashi, M. & Pascual-Leone, A. (2003) Lancet Neurol 2, 145-156], the ability to silence the primate brain with the onset and offset times of milliseconds (i.e. the timescale of action potentials in the brain) has not yet been demonstrated.

Experimental implementation of the invention has demonstrated that, while Halo requires blue light to recover it to its original state after prolonged yellow light illumination [Han, X. & Boyden, E. S. (2007) PLoS ONE 2, e299], Arch does not—after prolonged illumination, it recovers spontaneously in the dark. This key advantage is essential for prosthetics or other biotechnology applications in which multiple colors of light are neither optimal nor desired. For example, the ability to quickly repeat the silencing protocol with repeatable molecule efficacy enhances the capability to perform within-subject correlations. Shown in FIG. 6 are raw current trace of a neuron lentivirally infected with Arch, illuminated by a 15-s light pulse (575±25 nm, irradiance 7.8 mW/mm²) followed by 1-s test pulses delivered at 15, 45, 75, 105 and 135 s after the end of the 15-s light pulse, and population data of averaged Arch photocurrents (n=11 neurons) sampled at the times indicated by the vertical dotted lines that extend into the top trace.

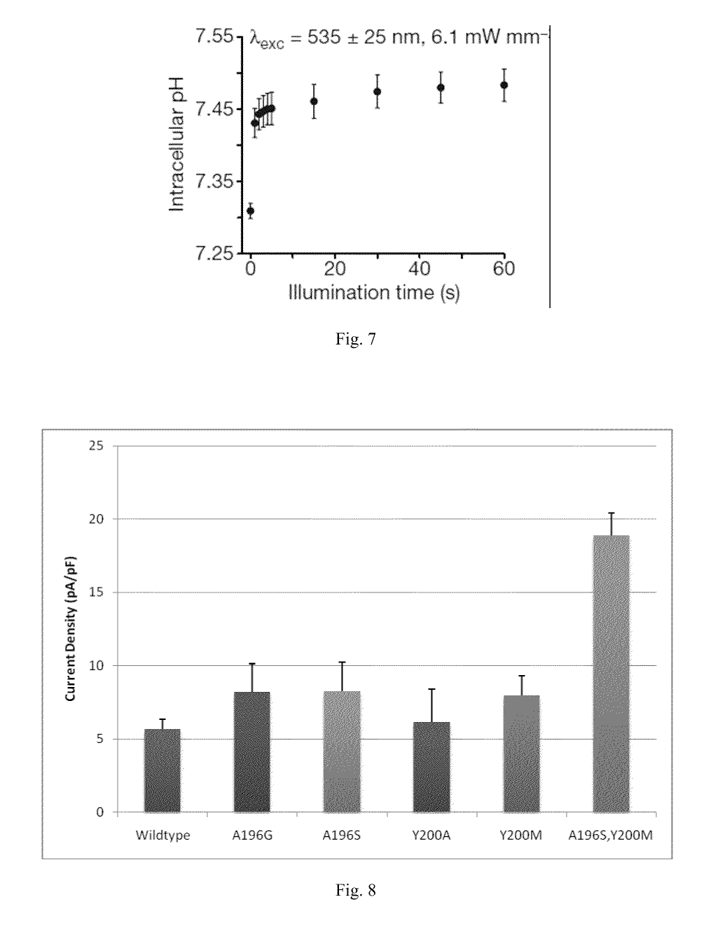

Proton pumps were also employed according to one aspect of the present invention to alter intracellular pH using light. Shown in FIG. 7 are intracellular pH measurements in neurons expressing Arch over a 1-min period of continuous illumination and simultaneous imaging (535±25 nm light, 6.1 mW/mm²) using SNARF-1 pH-sensitive ratiometric dye (n=10-20 cells per data point). This result also demonstrates that silencing neural activity via light-driven proton pumps leads to controllable pH changes that are on the order of normal neural activity [Chesler, M. (2003) Physiol Rev 83, 1183-1221; Kaila, K. & Ransom, B. R. (1998) pH and brain function (Wiley-Liss, New York] as a measure of safety. The simultaneous alteration of pH and membrane potential can be utilized for a combined therapeutic effect, such as simultaneous treatment of cortical spreading depression and its accompanying acidification that are commonly observed in migraine, stroke, and ischemia [Parsons, A. A. & Strijbos, P. J. (2003) Curr Opin Pharmacol 3, 73-77; Gault, L. M., Lin, C.-W., LaManna, J. C., & David Lust, W. (1994) Brain Research 641, 176-180].

Another aspect of the invention includes enhancements to the functional performance of the heterologously expressed proton pumps in mammalian cells via site-directed mutagenesis. The performance of these example compositions of matter may be altered by site-directed mutagenesis, such as the A196S+Y200M double mutation to Mac that leads to 3.3-fold improvement in photocurrent density. The performance of the above may also be altered by appending N-terminal and C-terminal peptide sequences to affect cellular trafficking, such as the N-terminal prolactin [Jungnickel, B. & Rapoport, T. A. (1995) Cell 82, 261-270] endoplasmic sorting sequence (denoted as ‘PRL’) (amino acid sequence: SEQ ID No 7; DNA sequence: SEQ ID No 8), or the MHC class I antigen signal sequence (denoted as “ss”) from reference [Munoz-Jordan, J. L., Laurent-Rolle, M., Ashour, J., Martinez-Sobrido, L., Ashok, M., Lipkin, W. I., & Garcia-Sastre, A. (2005) J. Virol. 79, 8004-8013] (amino acid sequence: SEQ ID No 9; DNA sequence: SEQ ID No 10), or, the human cytochrome c oxidase VIII N-terminal mitochondrial targeting sequence (denoted as “MTS8”) (amino acid sequence: SEQ ID No 11; DNA sequence: SEQ ID No 12) [Rizzuto, R., Nakase, H., Darras, B., Francke, U., Fabrizi, G. M., Mengel, T., Walsh, F., Kadenbach, B., DiMauro, S., & Schon, E. A. (1989) Journal of Biological Chemistry 264, 10595-10600; Rizzuto, R., Brini, M., Pizzo, P., Murgia, M., & Pozzan, T. (1995) Current Biology 5, 635-642]), or combinations thereof, as exemplified by the ss-Prl-Arch (i.e. ss::prl::Arch fusion) molecule reported in Chow, B. Y., Han, X., Dobry, A. S., Qian, X., Chuong, A. S., Li, M., Henninger, M. A., Belfort, G. M., Lin, Y., Monahan, P. E., et al. (2010) Nature 463, 98-102 (Genbank accession # GU045597), or ss-Prl-Arch-GFP (Genbank accession # GU045599).

FIG. 8 is a graph showing peak current density recorded from Mac mutants using whole-cell patch clamp that enhance photocurrent generation, where “wild type” denotes Mac (leptosphaeria maculans rhodopsin). As shown in FIG. 8, mutants were expressed in HEK293FT cells and illuminated with 575±25 nm light at 7.8 mW/mm² irradiance. The A196S+Y200M double mutant had a 3.3-fold increase in photocurrent than wild type Mac, under the stated conditions.

One aspect of the present invention is the creation of a class of bi-directional control molecules, exemplified by the W96Y-like mutants of archaerhodopsins. Such single molecules can be used as shunt-like molecules, where the inhibitory photocurrent is limited by the voltage dependence of the molecule's multiple modes of ion translocation for more naturalistic and controlled neural silencing (e.g. may avoid triggering hyperpolarization-dependent depolarizing currents).

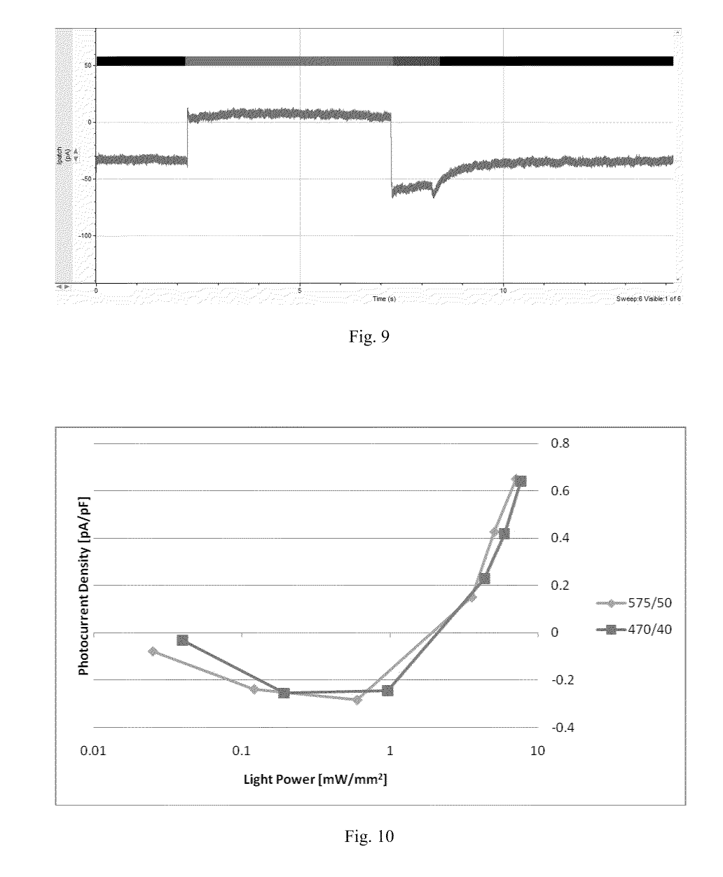

In this aspect of the present invention, bi-directional proton pumps were engineered from archaerhodopsins, created by the analogous W96Y mutation of H. salinarum bacteriorhodopsin [Marti, T., Otto, H., Mogi, T., Rosselet, S. J., Heyn, M. P., & Khorana, H. G. (1991) J Biol Chem 266, 6919-6927; Mogi, T., Stern, L. J., Chao, B. H., & Khorana, H. G. (1989) J Biol Chem 264, 14192-14196; Mogi, T., Stern, L. J., Marti, T., Chao, B. H., & Khorana, H. G. (1988) Proc Natl Acad Sci USA 85, 4148-4152] (from here on, this analogous position and mutation will be known as “W96Y-like”. FIG. 9 is a raw current trace recorded in a HEK cell expressing ArchT(w), illuminated with orange light (orange dash, 607±36 nm), followed by near-ultraviolet light (purple dash, 436±20 nm), thus demonstrating the potentiality for bi-directional control of membrane voltage using two different colors of light to address one molecule.

The direction of proton translocation via these molecules can be affected by color, intensity of illumination, or a combination of the two. The corresponding mutant of ArchT will be denoted as ArchT(w). Other archaerhodopsins from H. sodomense, Halorubrum strain aus-1 and Halorubrum strain aus-2 [Mukohata, Y., Ihara, K., Tamura, T., & Sugiyama, Y. (1999) J Biochem 125, 649-657; Ihara, K., Umemura, T., Katagiri, I., Kitajima-Ihara, T., Sugiyama, Y., Kimura, Y., & Mukohata, Y. (1999) J Mol Biol 285, 163-174; Enami, N., Yoshimura, K., Murakami, M., Okumura, H., Ihara, K., & Kouyama, T. (2006) J Mol Biol 358, 675-685] also resulted in this bi-directional functionality, whereas mutations to other microbial rhodopsins such as the H. salinarum bacteriorhodopsin, Mac, and channelrhodopsin [Boyden, E. S., Zhang, F., Bamberg, E., Nagel, G., & Deisseroth, K. (2005) Nature neuroscience 8, 1263-1268], respectively, did not lead to such bi-directional molecules, and thus this engineered functionality is non-obvious to one of ordinary skill in the art.

FIG. 10 is a plot depicting bi-directional optical control of an archaerhodopsin “W96Y-like” variant derived from Halorubrum strain aus-2, similar to the ArchT(w) variant of Halorubrum strain TP009. Here the direction of ion-translocation is determined by illumination intensity regardless of wavelength (here shown for 470±20 nm and 575±25 nm illumination). In FIG. 10, light power is plotted on a logarithmic scale.

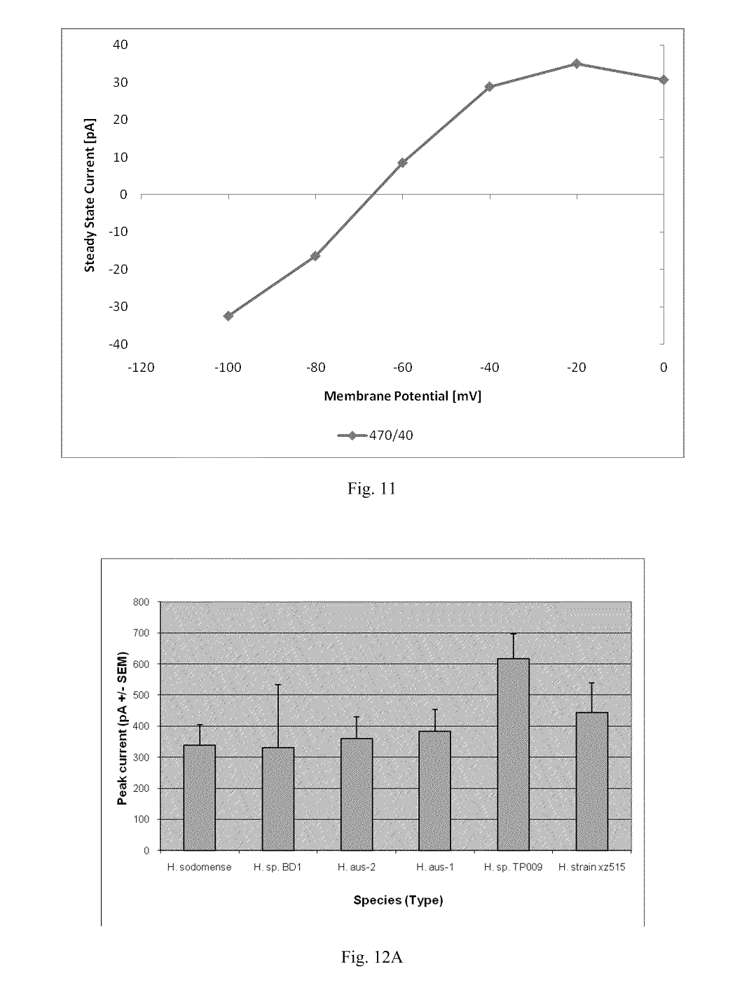

FIG. 11 is a plot depicting optically induced shunt-like activity exhibited by an archaerhodopsin “W96Y-like” variant derived from Halorubrum strain aus-1, similar to the ArchT(w) variant of Halorubrum strain TP009. When a HEK cell is illuminated with 470±20 nm light at 8 mW/mm², the reversal potential is between −65 and −70 mV.

Related to the W96Y-like mutants of archaerhodopsins is the variable control of membrane conductance, with respect to optical illumination wavelength, intensity, and transmembrane voltage. Unlike naturally occurring bi-directional molecules that function in mammalian cells, such as fSR's, these engineered variants have sustained steady state currents. The other known bi-directional molecules to date, proteorhodopsins, do not function under mammalian physiological conditions, as assessed in HEK cells and mouse primary neuron culture with the proteorhodopsins commonly known as BAC31A8, Hot75m4, and PalE6 [Beja, O., Spudich, E. N., Spudich, J. L., Leclerc, M., & DeLong, E. F. (2001) Nature 411, 786-789; Kelemen, B. R., Du, M., & Jensen, R. B. (2003) Biochim Biophys Acta 1618, 25-32; Kim, S. Y., Waschuk, S. A., Brown, L. S., & Jung, K. H. (2008) Biochim Biophys Acta 1777, 504-513].

In another aspect of the present invention, proton pumps are targeted to organelles in multi-cellular systems, multi-cellular organisms, and mammalian cell lines. In an exemplary implementation, ArchT was successfully targeted to mitochondria of mammalian HEK cell lines via the appendage of the human cytochrome c oxidase VIII N-terminal mitochondrial targeting sequence. The ability to augment proton gradients across the mitochondrial membrane with light enhances ATP production similar to as performed at the whole single-cell organism level with E. coli [Walter, J. M., Greenfield, D., Bustamante, C., & Liphardt, J. (2007) Proceedings of the National Academy of Sciences 104, 2408-2412]; the conversion of light to metabolizable forms of energy may decrease the innate dependence of the cell on glucose-dependent energy production and oxidative phosphorylation that can generate harmful reactive oxygen species and oxidative stress. Additional examples of mitochondrial targeting sequences include, but are not limited to TOM70, NADH ubiquinone oxoreductase, aldehyde dehydrogenase-2, ATP synthase alpha-subunit, and mitochondrial ATP-ase inhibitor.

One aspect of the invention uses light-activated proton pumps to hyperpolarize neurons. Light-activated microbial proton pumps have significantly improved kinetics over their chloride counterparts (e.g., N. pharaonis halorhodopsin) because they lack the long-lived inactive states, and in the case of proton-pumping archaerhodopsins versus halorhodopsins from various classes of species (bacteria, halobacter, haloarcula, and halorubrum chloride pumps), are demonstrably faster [Chow, B. Y., Han, X., Dobry, A. S., Qian, X., Chuong, A. S., Li, M., Henninger, M. A., Belfort, G. M., Lin, Y., Monahan, P. E., et al. (2010) Nature 463, 98-102]. This enables more temporally precise silencing, as well as more consistent long-term silencing for neural prosthetics and treatments of disease. Many disorders are disorders of neural excitability; the ability to shut down specific kinds of cells would greatly enhance the treatment of them.

Leptosphaeria maculans rhodopsin is blue-activatable, and thus allows hyperpolarization of cells with a color of light heretofore not used in biotechnology for hyperpolarization of cells. By using Mac and Halo together, hyperpolarization of two different populations of cells in the same tissue or in the same culture dish becomes possible. This simultaneous, two-color inactivation, is particularly promising for complex tissues such as the brain.

The use of other functional classes of molecules was enabled in mammalian cells during the screening process, including but not limited to: cruxhalorhodopsins (chloride pumps from Haloarcula) that have red-shifted action spectrum from others tested (e.g. see supplementary table 2 of Chow, B. Y., Han, X., Dobry, A. S., Qian, X., Chuong, A. S., Li, M., Henninger, M. A., Belfort, G. M., Lin, Y., Monahan, P. E., et al. (2010) Nature 463, 98-102), archaerhodopsins and cruxrhodopsins (proton pumps from Halorubrum and Haloarcula, respectively) as particularly efficacious silencers of neural activity that traffic well to mammalian membranes, and Acetabularia acetabulum as an exemplar of algal rhodopsins [Tsunoda, S. P., Ewers, D., Gazzarrini, S., Moroni, A., Gradmann, D., & Hegemann, P. (2006) Biophys J 91, 1471-1479], which are blue-shifted from proton pumps from the archaeal kingdom [Saranak, J. & Foster, K. W. (2005) Eukaryotic Cell 4, 1605-1612].

The performance of these molecules or classes of molecules can be tuned for optimal use, particularly in context of their use in conjunction with other molecules or optical apparatus. For example, in order to achieve optimal contrast for multiple-color silencing, one may desire to either improve or decrease the performance of one molecule with respect to one another, by the appendage of trafficking enhancing sequences or creation of genetic variants by site-directed mutagenesis, directed evolution, gene shuffling, or altering codon usage. Molecules or classes of molecules may have inherently varying spectral sensitivity that may be functionally advantageous in vivo (where scattering and absorption will vary with respect to wavelength, coherence, and polarization), by tuning the linearity or non-linearity of response to optical illumination with respect to time, power, and illumination history.

One particular aspect of the invention is the use of the halorubrum genus of haloarchaea. These have been identified as particularly efficacious light-activated proton pumps because they express particularly well in mammalian membranes and perform robustly under mammalian physiological conditions, based on the shown characterization of every full archaerhodopsin clone known at the time of initial disclosure.

FIG. 12A is a histogram of photocurrents measured in (mouse hippocampus) cultured neurons expressing all known electrogenic archaerhodopsin full sequence clones (at the time of disclosure), demonstrating that the class of proton pumps from halorubrum perform exceptionally well under mammalian physiological conditions and produce photocurrents >2.5× greater than the state of the art naturally occurring gene product (at the time of the disclosure). FIG. 12B is a confocal fluorescence image of cultured neuron expressing Arch with a GFP fused to the C-terminus (scale bar=20 um) showing good membrane localization in the absence of the appended signal sequences (i.e. the naturally occurring gene product or protein sequence). It should be noted that H. lacusprofundiopsin was shown to be non-electrogenic in its primary physiological role, bearing semblance to fSR's, and thus it is not shown.

Another aspect of the present invention is the enablement of systematic tuning of membrane trafficking properties by appending, deleting, or replacing, small peptide sequences. For example, the prolactin (PRL) signal sequence boosts Arch photocurrents by ˜34%, while the ER2 sequence (C-terminal ER export sequence from KiR2.1) [Stockklausner, C. & Klocker, N. (2003) J Biol Chem 278, 17000-17005] reduces intracellular blebbing but does not increase the photocurrent; the effects of the two sequences can therefore be systematically combined for an trafficking- and photo-current improved variant, hereby termed sp-Arch-ER2, as an example and preferred embodiment in the form of signal sequence::PRL::“product”::ER2, where “product” is a genetically encoded gene product.

FIG. 13 is a histogram depicting the cumulative effect of appending signal sequences to a naturally occurring protein sequence. As shown in FIG. 13, intracellular blebs or puncta are reduced by the appendage of an N-terminal “ss” and the C-terminal “ER2” sequence as has been previously reported, but do not enhance the photocurrent density. The addition of the “ss” and “PRL” sequences to the N-terminus improves the current density by 34%. The Arch variant bearing all 3 moieties has reduced intracellular agglomeration of protein (conferred by the ss+ER2 combination) and improved photocurrent density like the ss-PRL-Arch variant.

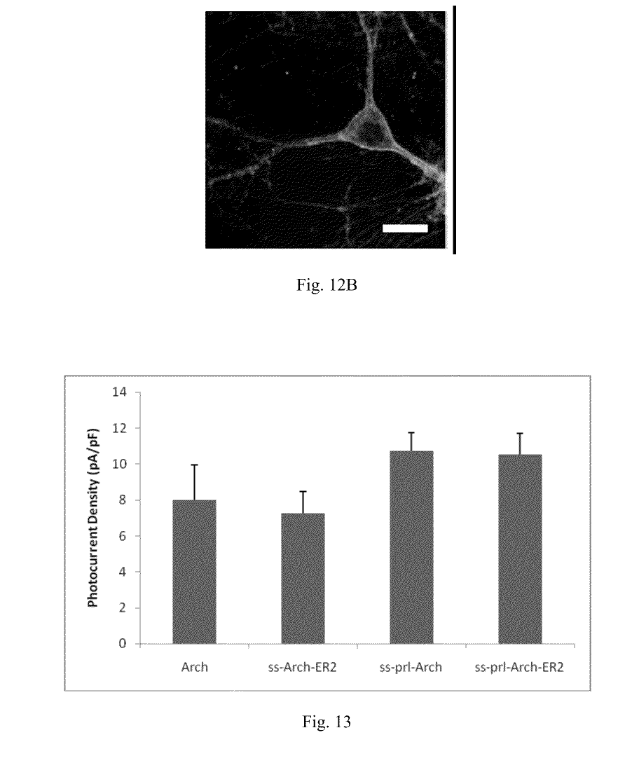

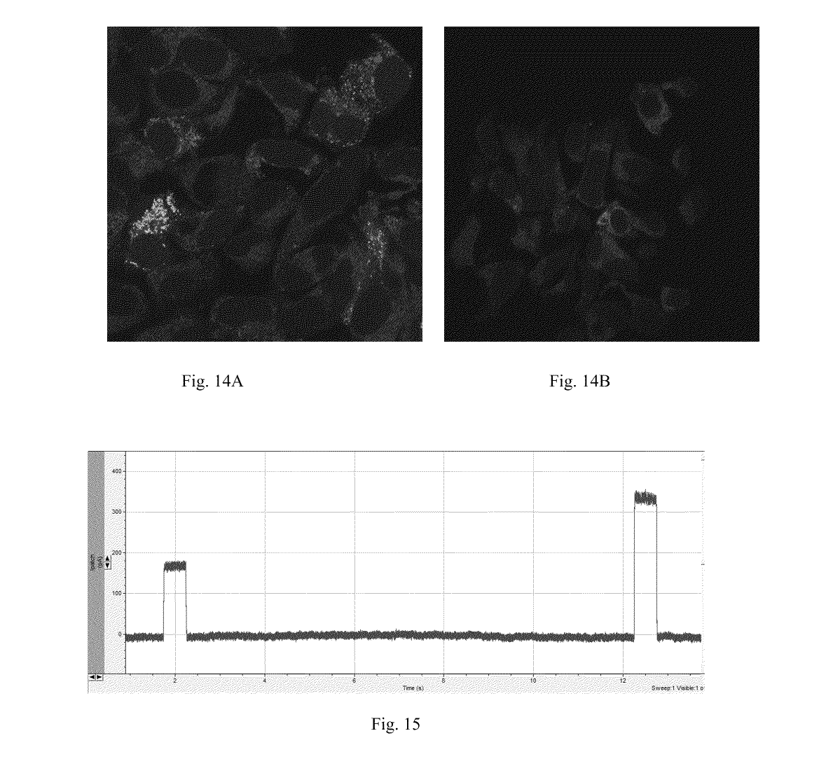

One application of the invention is the targeting of proton pumps to specific organelles. For example, proton pumps targeted to the mitochondria can augment or diminish the ATP-production capability of the cell, and thus affect cell metabolism. A related aspect of the invention is the targeting of proton pumps to organelles to affect the physiology of multi-cellular organisms. In an exemplary implementation of this application, FIGS. 14A and B are fluorescence images showing HEK293 cells cultured on coverslips that were transfected with MTS8-GFP (FIG. 14A) and MTS8-ArchT-GFP (FIG. 14B) plasmids. Cultures were then stained with Mitotraker Red CMXRos (Invitrogen) on post-transfection day 2 and fixed in 4% paraformaldehyde before being mounted on a glass slide for confocal imaging.

As previously described, an aspect of the invention is the use of the proton pumping rhodopsin genes from eukaryotes, such as algae or fungus. Besides Mac, another fungal rhodopsin that has been identified as being a particularly efficacious light-activated proton pump is S. sclerotorium, a fungal opsin that has never been characterized before physiologically. FIG. 15 is a trace of the S. sclerotorium opsin in a HEK cell demonstrating that it too is efficacious. As shown in FIG. 15, a voltage clamped HEK293 cell expressing the S. sclerotorium proton pump exhibits photocurrents upon illumination with blue (470 nm) and yellow light (575 nm).

Another aspect of the invention is the use of inwardly rectifying proton pumps. For example, a molecule like a transducer-free sensory rhodopsin [Sineshchekov, O. A., Sasaki, J., Phillips, B. J., & Spudich, J. L. (2008) Proc Natl Acad Sci USA 105, 16159-16164; Chen, X. & Spudich, J. L. (2004) J Biol Chem 279, 42964-42969] exhibits bi-directional proton transport at two different wavelengths, and thus can be used to depolarize or hyperpolarize a cell using the same protein, as well as treat acidosis or alkalinosis with the same protein.

Another aspect of the invention is the generation of locally acidic or alkaline environment proximal to the light-activated proton pump, thereby affecting the binding of membrane receptors to their targets (for example, acid-sensing ion channels) by modulating pH. Such a method may be used to alter the efficacy of pH dependent pharmacological agents, such as the treatment of cancer where the tumor environment is highly acidic, or for the deprotection of pharmacological agents bearing acid- or base-labile protecting groups.

In one embodiment of the invention, sensitizing chromophores, such as chlorophyll or salinixanthin [Balashov, S. P., Imasheva, E. S., Boichenko, V. A., Anton, J., Wang, J. M., & Lanyi, J. K. (2005) Science 309, 2061-2064], are used to broaden or shift the absorbance spectrum of the molecule, and are particularly advantageous for multi-color silencing, tuning the absorbance for optimality with specific optical apparatus (e.g. narrow excitation LEDs and lasers, long wavelength absorption for better transmission through tissue, etc.), or the creation of harmful UV oxidized species.

Another aspect of the invention is the identification of the analogous amino acid resides to the retinal flanking S141, M145, and P186 residues in H. salinarum bacteriorhodopsin (for example, as numbered in Marti, T., Otto, H., Mogi, T., Rosselet, S. J., Heyn, M. P., & Khorana, H. G. (1991) J Biol Chem 266, 6919-6927; Mogi, T., Stern, L. J., Chao, B. H., & Khorana, H. G. (1989) J Biol Chem 264, 14192-14196; Mogi, T., Stern, L. J., Marti, T., Chao, B. H., & Khorana, H. G. (1988) Proc Natl Acad Sci U S A 85, 4148-4152) as mutagenesis targets for systematic spectral tuning. Mutagenesis to these residues in ArchT and Mac more easily resulted in altered spectral responses at higher and lower energies of the peak absorption (by boosting or reducing the higher energy or lower energy responses, independently or in concert) often without decrease in physiological function, when compared to other retinal flanking positions identified with previously reported molecules. For example, the S151G mutant of ArchT (analogous to S141 residue above mentioned) blue-shifted the peak absorption of the wild type molecule by 18 nm, and the A196+Y200M double mutant of Mac (corresponding to the S141+M145 residues mentioned above) red-shifted the wild-type Mac's peak absorption by 21 nm.

Another aspect of the invention is the reduction to practice of technologically viable deep brain silencers or deep brain inhibitors (hereby termed DBSi or DBI, respectively) in the primate brain. Deep brain silencing may be used in conjunction with, deep brain stimulation, for example, to limit adverse side effects created by electrical stimulation that affects all cell types.

Another aspect of the invention is the method of coupling the molecule's efficacy to the selection pressure or screening criteria in directed evolution. For example, if the molecule is screened in an archaebacteria devoid of a microbial rhodopsin that it utilizes for phototrophy, then species bearing poorly performing molecules under the test conditions will not survive. Such coupling of performance to selection enables autonomous and label-free screening.

Another aspect of the invention are various compositions of matter that have been reduced to practice, including plasmids encoding for the above genes, especially lentiviruses carrying payloads encoding for the above genes, adeno-associated viruses carrying payloads encoding for the above genes, cells expressing the above genes, animals expressing those genes (including mice, but implicating primates and humans).

In one application of the invention, cells are hyperpolarized, thus activating endogenous hyperpolarization-activated conductances (such as T-type calcium channels and Ih currents), and then drugs are applied that modulate the response of the cell to hyperpolarization (using a calcium or voltage-sensitive dye). This enables new kinds of drug screening using just light to activate the channels of interest, and using just light to read out the effects of a drug on the channels of interest.

Another application of the invention is the use of light-activated proton pump to increase the pH of the cell. Such a technique may be used to treat acidosis, particularly as a method of neuroprotection during traumatic brain injury.

In yet another application, light-activated proton pumps are employed for the coupled effects of hyperpolarization and intracellular alkalinization. For example, both phenomena can induce spontaneous spiking in neurons by triggering hyperpolarization-induced cation currents or pH-dependent hyper-excitability.

Other applications of the invention include, but are not limited to, use of proton pumps of microbial origin as an exemplary test system for assessing membrane protein trafficking and physiological function in heterologously expressed systems, generation of sub-cellular voltage or pH gradients, particularly at synapses and in synaptic vesicles to alter synaptic transmission, and mitochondria to improve ATP synthesis, and use of light-activated proton pumps to hyperpolarize a cell that has high intracellular chloride concentrations, such as young adult-born neurons.

The invention and its uses are envisioned to cover one or more of the following: The use of light activated proton pumps to adjust the voltage potential of cells, sub-cellular regions, and extracellular regions. The use of light activated proton pumps to adjust the pH of cells, sub-cellular regions, and extracellular regions. The use of light activated proton pumps to release protons as chemical transmitters. These methods wherein the proton pump is a microbial rhodopsin that is outwardly rectifying, is from the halorubrum genus of archaeabacteria, is leptosphaeria maculans, P. triticirepentis, or S. scelorotorium, or is from the genus Acetabularia, and specifically halorubum strain aus-1, halorubrum strain aus-2, halorubrum sodomense, halorubrum strain BD1, halorubrum stain xz515, halorubrum strain TP009, halorubrum lacusprofundi, or Acetabularia acetabulum, leptosphaeria maculans, pyrenophora tritici-repentis, or sclerotinia sclerotorium.

These methods are used particularly for neural silencing, that is, hyperpolarizing a neuron to prevent it from depolarizing, spiking, or otherwise signaling (e.g., to release neurotransmitters). The use of two or more light-activated membrane proteins for multi-color neural silencing (e.g., Ace or Mac, and Halo), expressing them in different populations of cells in a tissue or in a dish, and the illuminating them each with different colors of light. The use of light-activated proton pumps instead of chloride channels to improve the regeneration speed of the active pumping state of hyperpolarizing electrogenic membrane proteins. The method wherein the proton pump is a microbial rhodopsin that in itself is both inwardly and outwardly rectifying at two different colors of light. The use of light-activated proton pumps to treat cellular and muscle acidosis or alkalinosis. The use of light-activated proton pumps to treat acidosis and alkalinosis in the cardiac system. The use of light-activated proton pumps for neuroprotection after traumatic brain injury. The use of light-activated proton pumps to modify cellular pH, which then modifies cellular potential, or vice versa. The use of light-activated proton pumps to generate pH and electrogenic gradients in vesicles to mediate their transport, fusion with membranes, or incorporation of molecules. The use of light-activated proton pumps to treat pain or irritation induced by molecular interactions with acid- or alkaline-sensitive ion channels or receptors. The use of light-activated proton pumps to hyperpolarize cells that have high intracellular chloride content. The use of light-activated proton pumps to modify pH for the purpose of affecting cellular, multi-cellular, or organismal development.

The ability to optically perturb, modify, or control cellular function offers many advantages over physical manipulation mechanisms, such as speed, non-invasiveness, and the ability to easily span vast spatial scales from the nanoscale to macroscale. One such approach is an opto-genetic approach, in which heterologously expressed light-activated membrane proteins move ions with spectral selectivity, as well as potential ion-selectivity and cell-type specificity, the latter by way of promoter-targeting [Han, X. & Boyden, E. S. (2007) PLoS ONE 2, e299; Zhang, F., Wang, L. P., Brauner, M., Liewald, J. F., Kay, K., Watzke, N., Wood, P. G., Bamberg, E., Nagel, G., Gottschalk, A., et al. (2007) Nature 446, 633-639; Boyden, E. S., Zhang, F., Bamberg, E., Nagel, G., & Deisseroth, K. (2005) Nature neuroscience 8, 1263-1268]. To date, the light-activated cation channels channelrhodopsin-2 (ChR2) and volvox channelrhodopsin-1 (VChR1) have been demonstrated to depolarize neurons with millisecond resolution (i.e. the timescale of an action potential) [Boyden, E. S., Zhang, F., Bamberg, E., Nagel, G., & Deisseroth, K. (2005) Nature neuroscience 8, 1263-1268; Zhang, F., Prigge, M., Beyriere, F., Tsunoda, S. P., Mattis, J., Yizhar, O., Hegemann, P., & Deisseroth, K. (2008) Nature neuroscience 11, 631-633] by primarily triggering sodium influx into the cell. Likewise, the light-activated chloride pump NpHR has been used to silence neural activity by hyperpolarizing cells by way of chloride influx [Han, X. & Boyden, E. S. (2007) PLoS ONE 2, e299; Zhang, F., Wang, L. P., Brauner, M., Liewald, J. F., Kay, K., Watzke, N., Wood, P. G., Bamberg, E., Nagel, G., Gottschalk, A., et al. (2007) Nature 446, 633-639]. The light-activated G-protein coupled receptor RO4 has also been used to silence neural activity by way of decreasing pre-synaptic calcium conductance [Li, X., Gutierrez, D. V., Hanson, M. G., Han, J., Mark, M. D., Chiel, H., Hegemann, P., Landmesser, L. T., & Herlitze, S. (2005) Proc Natl Acad Sci USA 102, 17816-17821].

These approaches have already proven useful for applications like high-speed neural circuit mapping [Ayling, O. G., Harrison, T. C., Boyd, J. D., Goroshkov, A., & Murphy, T. H. (2009) Nat Methods; Wang, H., Peca, J., Matsuzaki, M., Matsuzaki, K., Noguchi, J., Qiu, L., Wang, D., Zhang, F., Boyden, E., Deisseroth, K., et al. (2007) Proc Natl Acad Sci USA 104, 8143-8148] and treating blindness in mice [Lagali, P. S., Balya, D., Awatramani, G. B., Munch, T. A., Kim, D. S., Busskamp, V., Cepko, C. L., & Roska, B. (2008) Nature neuroscience 11, 667-675]. However, the current state-of-the-art neural silencing tools have several shortcomings. Because the currently known light-activated inwardly rectifying chloride channels are kinetically limited by the fact that an inherent long-lived (t_{1/2}˜ten of minutes) inactive state exists in their photocycle such that their active chromophore cannot be regenerated. The G-protein coupled receptors are also limited by their speed, since a cascade of exogenous molecular events must occur before its activity can be re-established. Furthermore, the efficacy of the current state-of-the-art optical silencing methods lacks behind that of the state-of-the-art optical stimulation methods.

The reagents disclosed herein, and the class of molecules that they represent, enable: Significantly larger currents than any previous reagent (perhaps 3-5× higher under most conditions); Fast inhibition (far better than enabled by Li, X., Gutierrez, D. V., Hanson, M. G., Han, J., Mark, M. D., Chiel, H., Hegemann, P., Landmesser, L. T., & Herlitze, S. (2005) Proc Natl Acad Sci USA 102, 17816-17821); A new modality of control (involving H+ ions, not Cl− ions); Different spectra from older molecules (opening up multi-color control of cells); The ability to change pH of cells. Furthermore, with Mac, multiple-color silencing of multiple populations of cells in the same tissue or in the same culture dish is enabled for the first time. Mac-expressing cells will be hyperpolarizable using blue light, whereas Halo-expressing cells will be hyperpolarizable using yellow light; the two cells can sit side-by-side, and be individually addressed with different colors of light.

It is anticipated that the present invention will have numerous commercial applications. These include, but are not limited to, prosthetic applications such as, but not limited to, gene therapy+device applications in which excitable cells (heart cells, brain cells, etc.) can be silenced or have their pH altered, in order to produce long-term cell silencing for neural prosthetics and treatments of disease, which will likely be useful in the treatment of epilepsy, Parkinson's, and other disorders; drug screening applications including, but not limited to, hyperpolarizing cells, thus activating endogenous hyperpolarization-activated conductances (such as T-type calcium channels and I_h currents), and then applying drugs that modulate the response of the cell to hyperpolarization (using a calcium or voltage-sensitive dye); diagnostics applications, such as, but not limited to, sensitizing samples of tissues from patients to light, or converting them into other cell types (e.g., stem cells) and then sensitizing those to light; and the collection of optical energy, e.g. solar energy, using light-activated pumps expressed in cell lines.

Detailed methods. Novel opsin reagents: plasmid construction and lentivirus production. The opsins examined are listed in Table 1, which describes their molecule classes, species of origin, GenBank Accession numbers, and relevant references. Molecule classes chiefly include bacteriorhodopsins (proton pumps) and halorhodopsins (chloride pumps). Further sub-classifications of ion pump type denote the origin of the species: for example, a “cruxhalorhodopsin” is a chloride pump from the haloarcula genus of halobacteria. Opsins were mammalian codon-optimized, and were synthesized by Genscript (Genscript Corp., NJ). Opsins were fused in frame, without stop codons, ahead of GFP (using BamHI and Agel) in a lentiviral vector containing the CaMKII promoter, enabling direct neuron transfection, HEK cell transfection (expression in HEK cells is enabled by a ubiquitous promoter upstream of the lentiviral cassette), and lentivirus production (except for Halobacterium salinarum halorhodopsin, which was fused to GFP in the vector pEGFP-N3 (using EcoRI and BamHI) and only tested by transfection). eNpHR was synthesized as described before, by inserting the signaling sequence from the acetycholine receptor beta subunit (amino acid sequence: SEQ ID No 13; DNA sequence: SEQ ID No 14) at the N-terminus, and the ER2 sequence (amino acid sequence: SEQ ID No 15; DNA sequence: SEQ ID No 16) at the C-terminus of Halo. The ‘ss’ signal sequence from truncated MHC class I antigen corresponded to amino acid sequence SEQ ID No 9, DNA sequence SEQ ID No 10. The ‘Prl’ Prolactin signal sequence corresponded to amino acid sequence SEQ ID No 7, DNA sequence SEQ ID No 13. Halo point mutants for HEK cell testing were generated using the QuikChange kit (Stratagene) on the Halo-GFP fusion gene inserted between BamHI and EcoRI sites in the pcDNA 3.1 backbone (Invitrogen). All constructs were verified by sequencing, and codon-optimized sequences of key opsins were submitted to Genbank (mammalian codon-optimized Arch, GU045593; mammalian codon-optimized Arch fused to GFP, GU045594; mammalian codon-optimized Mac, GU045595; mammalian codon-optimized Mac fused to GFP, GU045596; ss-Prl-Arch, GU045597; ss-Arch-GFP-ER2, GU045598; ss-Prl-Arch-GFP, GU045599).

Replication-incompetent VSVg-pseudotyped lentivirus was produced as previously described, which allows the preparation of clean, non-toxic, high-titer virus (roughly estimated at ˜10⁹-10¹⁰ infectious units/mL). Briefly, HEK293FT cells (Invitrogen) were transfected with the lentiviral plasmid, the viral helper plasmid pΔ8.74, and the pseudotyping plasmid pMD2.G. The supernatant of transfected HEK cells containing virus was then collected 48 hours after transfection, purified, and then pelletted through ultracentrifugation. Lentivirus pellet was resuspended in phosphate buffered saline (PBS) and stored at −80° C. until further usage in vitro or in vivo.

Hippocampal and cortical neuron culture preparation, transfection, infection, and imaging. All procedures involving animals were in accordance with the National Institutes of Health Guide for the care and use of laboratory animals and approved by the Massachusetts Institute of Technology Animal Care and Use Committee. Swiss Webster or C57 mice (Taconic or Jackson Labs) were used. For hippocampal cultures, hippocampal regions of postnatal day 0 or day 1 mice were isolated and digested with trypsin (1 mg/ml) for ˜12 min, and then treated with Hanks solution supplemented with 10-20% fetal bovine serum and trypsin inhibitor (Sigma). Tissue was then mechanically dissociated with Pasteur pipettes, and centrifuged at 1000 rpm at 4° C. for 10 min. Dissociated neurons were plated at a density of approximately four hippocampi per 20 glass coverslips, coated with Matrigel (BD Biosciences). For cortical cultures, dissociated mouse cortical neurons (postnatal day 0 or 1) were prepared as previously described⁴⁰, and plated at a density of 100-200 k per glass coverslip coated with Matrigel (BD Biosciences). Cultures were maintained in Neurobasal Medium supplemented with B27 (Invitrogen) and glutamine. Hippocampal and cortical cultures were used interchangeably; no differences in reagent performance were noted.

Neurons were transfected at 3-5 days in vitro using calcium phosphate (Invitrogen). GFP fluorescence was used to identify successfully transfected neurons. Alternatively, neurons were infected with 0.1-1 μl of lentivirus per well at 3-5 days in vitro. Throughout the paper, neurons were transfected unless indicated as having been infected. All images and electrophysiological recordings were made on neurons 9-14 days in vitro (approximately 6-10 days after transfection or viral infection).

Confocal images of infected neurons in culture (briefly fixed in 4% paraformaldehyde) were obtained with a Zeiss LSM 510 confocal microscope (63× magnification objective lens). Culture images were maximum intensity projections made from sets of 5 images (1.0 μm image plane thickness) spaced along the z-axis by 0.5 micron steps. Quantitative confocal analysis of membrane expression of opsins was performed using infected neurons in culture (10 days post-infection, briefly fixed in 4% paraformaldehyde). Images were obtained with a Zeiss LSM 510 confocal microscope (63× magnification objective lens), always with the same illumination and observation parameters to avoid procedural variability. Given the near-100% viral infection rate, isolated neurons were chosen for analysis in order to reduce background fluorescence from nearby neurons and their processes. Images were analyzed in ImageJ (National Institutes of Health), based on a neuron-adapted version of a previously reported algorithm' used to assay membrane expression of channelrhodopsins and channelrhodopsin variants in HEK cells. An image was first filtered with a 2-pixel Gaussian blur, and the filtered image was subtracted from the original one to enhance the contour of the cell. These steps are exactly the same as those utilized before in HEK cells. However, because the membranes of neurons do not form simple shapes like the HEK cells as the original algorithm was designed for, black line sections were applied to separate the neuronal cell body from the processes. The magic wand can then accurately select the somatic membrane segments of a neuron, which can then be analyzed by the pixel intensity-value extraction method described for HEK cells. The value of the membrane fluorescence for a given neuron, reported in the text, was then defined as the area-weighted average of the line segments. While this method cannot prove that a given patch of membrane-proximal fluorescence exists exclusively on the outermost membrane (in principle a patch of fluorescence could reside just under the membrane), it does serve to discriminate between surface expression and ER retention, and has previously been validated in predicting functional physiological surface expression.

In vitro patch clamp recording and optical methods. Whole cell patch clamp recordings were made on neurons at 9-14 days in vitro, using a Multiclamp 700B amplifier, Digidata 1440 digitizer, and a PC running pClamp (Molecular Devices). During recording, neurons were bathed in Tyrode solution containing (in mM): 125 NaCl, 2 KCl, 3 CaCl₂, 1 MgCl₂, 10 HEPES, 30 glucose, 0.01 NBQX, and 0.01 gabazine, at pH 7.3 (NaOH adjusted), and with 305-310 mOsm (sucrose adjusted). Borosilicate glass (Warner) pipettes were filled with a solution containing (in mM): 125 K-Gluconate, 8 NaCl, 0.1 CaCl₂, 0.6 MgCl₂, 1 EGTA, 10 HEPES, 4 Mg-ATP, 0.4 Na-GTP, at pH 7.3 (KOH adjusted), and with 295-300 mOsm (sucrose adjusted). Pipette resistance was 5-10 MΩ; access resistance was 10-30 MΩ, monitored throughout the voltage-clamp recording; resting membrane potential was ˜60 mV in current-clamp recording.

For ion selectivity tests, neurons were bathed in chloride-free recording solution containing (in mM): 125 Na-Gluconate, 2 K-Gluconate, 3 CaSO₄, 1 MgSO₄, 10 HEPES, 30 glucose, 0.01 NBQX, 0.01 gabazine, at pH 7.3 (NaOH adjusted), and with 305-310 mOsm (sucrose adjusted), or potassium-free recording solution containing (in mM): 125 NaCl, 2 CsCl, 3 CaCl₂, 1 MgCl₂, 10 HEPES, 30 glucose, 0.01 NBQX, 0.01 gabazine, at pH 7.3 (NaOH adjusted), 305-310 mOsm (sucrose adjusted). During these ion selectivity tests, pipettes were filled with chloride-free pipette solution containing (in mM): 125 K-Gluconate, 8 Na-Gluconate, 0.1 CaSO₄, 0.6 MgSO₄, 1 EGTA, 10 HEPES, 4 Mg-ATP, 0.4 Na-GTP, pH 7.3 (KOH adjusted), 295-300 mOsm (sucrose adjusted), or potassium-free pipette solution containing (in mM): 125 Cs-methanesulfonate, 8 Na-Gluconate, 0.1 CaSO₄, 0.6 MgSO₄, 1 EGTA, 10 HEPES, 4 Mg-ATP, 0.4 Na-GTP, pH 7.3 (CsOH adjusted), 295-300 mOsm (sucrose adjusted). During resting membrane potential shifting, neurons were bathed in recording solution containing (in mM): 125 N-methyl-D-glucamine, 2 Cs-methanesulfonate, 3 CdSO₄, 1 MgSO₄, 10 HEPES, 30 glucose, 0.01 NBQX, 0.01 gabazine, pH 7.3 (H₂SO₄ adjusted), 305-310 mOsm (sucrose adjusted), and pipettes were also filled with analogous solutions containing (in mM): 125 Cs-methanesulfonate, 8 N-methyl-D-glucamine, 0.1 CdSO₄, 0.6 MgSO₄, 1 EGTA, 10 HEPES, 4 Mg-ATP, 0.4 Tris-GTP, pH 7.3 (CsOH adjusted), 295-300 mOsm (sucrose adjusted).

Photocurrents were measured with 1-second or 15-second duration light pulses in neurons voltage clamped at −60 mV. Light-induced membrane hyperpolarizations were measured with 1-second light pulses, in neurons current clamped at their resting membrane potential. For all experiments except for the action spectrum characterization experiments, a DG-4 optical switch with 300 W xenon lamp (Sutter Instruments) was used to deliver light pulses. The DG-4 was controlled via TTL pulses generated through a Digidata signal generator. A 575±25 nm bandpass filter (Chroma) was used to deliver yellow light, and a 535±25 nm filter was used to deliver green light. For selective activation of Halo versus Mac at different wavelengths, a 470±20 nm bandpass filter (Chroma) was used to deliver blue light (0.92 mW/mm², through a 40× objective), and a 630±15 (Chroma) was used to deliver red light (2.6 mW/mm²). For action spectrum characterization (Table 2), a Till Photonics PolyChrome V, 150 W Xenon, 15 nm monochromator bandwidth, was used.

Data was analyzed using Clampfit (Molecular Devices) and MATLAB (Mathworks, Inc.). Statistical analysis and curve fitting was done with Statview (SAS Institute), MATLAB, or Origin (OriginLab). Reported action spectra are second-order Gaussian fits (performed in MATLAB), because action spectra were asymmetric, with a broad “shoulder” at wavelengths shorter than the primary peak wavelength.

For the initial screening of photocurrents, yellow light (575±25 nm, 7.8 mW/mm², through a 40× lens) was chiefly used (see below for exceptions); accordingly, in order to adjust the screen data to reflect the photocurrent for each molecule that would have been observed at its respective spectral maximum, photocurrents were spectrum normalized by calculating the overlap integral between the second-order Gaussian fit of the action spectrum for each molecule and the passband of the yellow illumination filter used for the screen (or in other words, integrating the Gaussian fit between 550 and 600 nm), and then dividing that value by the integral of the whole action spectrum for that molecule. These resultant ratios, or “Spectral Screen Normalization Factors,” are summarized (normalized to that ratio for Halo itself) in the rightmost column of Table 2.

In the cases of gPR, bPR, and the Leptosphaeria maculans (Mac, LR, Ops) and Acetabularia acetabulum (Ace, AR) proton pump opsins, green light (535±25 nm, 9.4 mW/mm², through a 40× lens) was used during the screen, due to the significantly blue-shifted action spectrum of these genes. These four spectra were also normalized to the respective spectral maxima of each molecule, as described above, as well as weighted by the output power of the lamp. All screen photocurrents and spectra were measured in neurons except for the action spectrum of Ace, which was recorded in HEK 293FT cells for better resolution (due to the extremely small currents of Ace in neurons).

In order to extend the power characterization of Arch beyond the power of the yellow light available with the microscope and configuration that employed (7.8 mW/mm² irradiance, through a 40× lens), extrapolation to higher effective yellow powers was accomplished by equating various powers of unfiltered white light illumination from the DG4, to approximate effective yellow power equivalents. These effective irradiances were estimated by adjusting the output power of unfiltered white light from the DG4, and comparing the photocurrents vs. those generated with 575±25 nm yellow light in the same Arch-expressing neuron, at low light powers, until the photocurrent magnitudes were similar (p>0.7, paired t-test; N=6). In support of this method for estimating effective irradiances, no noticeable photocycle-accelerating effects of non-yellow light were observed for Arch. Light power-photocurrent curves, thus estimated, were fitted with a Hill plot. To compare to Arch, Halo currents measured for the dose response experiment were obtained using a Halo variant that demonstrated similar photocurrent densities compared to unmodified Halo (p>0.7, t-test; N=16), bearing a N-terminal signal sequence from a truncated MHC class I antigen (SEQ ID NO 9) and the C-terminal golgi export sequence from bovine rhodopsin (SEQ ID NO 17); these measurements were then scaled by the photocurrent ratio between Halo and this variant measured at 7.8 mW/mm².

HEK cell culture, transfection, and electrophysiology. HEK 293FT cells (Invitrogen) were maintained in DMEM medium (Cellgro) supplemented with 10% fetal bovine serum (Invitrogen), 1% penicillin/streptomycin (Cellgro) and 1% sodium pyruvate (Biowhittaker)). For recording, cells were plated at 5-10% confluence on uncoated glass coverslips, where they adhered to surfaces typically within 12-18 hours. Adherent cells were transfected using TransIT 293 transfection kits (Minis). Cells were recorded by whole-cell patch clamp 1.5-2 days later, as described above for neurons, except that they were voltage clamped at −40 mV, with a Tyrode bath solution lacking GABAzine and NBQX.

Intracellular pH imaging. Intracellular pH (denoted pHᵢ) imaging was performed using a cell-permeant ratiometric fluorescent dye, carboxy-SNARF-1 AM ester (Invitrogen), based on previously reported methods. In order to eliminate background fluorescence that would interfere with pH, imaging using SNARF-1, the fusion protein comprising Arch and cyan fluorescent protein (CFP) was used. The DG-4 was used to deliver light pulses (6.1 mW/mm², through a 20× lens) via a green 535±25 nm bandpass filter (Chroma). Neurons were loaded with 10 μM SNARF-1-AM ester in Tyrode solution for 10 minutes, and then washed twice with Tyrode. Arch-expressing neurons were identified by their CFP fluorescence (Chroma CFP set, λ_{excitation}=436±20 nm, λ_{dichroic}=455 nm, λₑₘᵢₛₛᵢₒₙ=480±20 nm). After waiting 1 additional minute, Arch and the SNARF-1 dye were simultaneously excited with 535±25 nm light, and the dye was imaged near the isosbestic point of the dye for 500 ms using a 610±37.5 nm bandpass filter (k_{dichroic}=565 nm, Chroma) to obtain a baseline SNARF loading level. After waiting another minute for the neuron to recover its initial pHᵢ, the neuron and dye were again excited with green light, and the dye was imaged at various time points with 1 second exposure lengths, using a 640±25 nm bandpass filter (λ_{dichroic}=600 nm, Chroma). Arch-negative neighboring neurons in the same field of view were imaged to provide a basis for comparison, and also to provide baseline pH of the cells as a point of reference, as done in ref⁴¹. Immediately after this period, the dye was again imaged near the isobestic point for 500 ms to assess for photo-bleaching or dye leakage; no change was observed (p>0.7 comparing before vs. after 60 seconds of illumination; n=5 neurons).

Calibration of the dye was performed by the “high K⁺/nigericin” method⁴², in which cells were immersed in a high K⁺ Tyrode-like solution containing (in mM): 125 KCl, 2 NaCl, 3 CaCl₂, 1 MgCl₂, 10 HEPES, 30 glucose, 0.01 NBQX, 0.01 gabazine, 0.001 TTX, pH 5.5 and pH 8.5 initial stock solutions (KOH adjusted), 305-310 mOsm (sucrose adjusted). The calibration curve was taken between pH 6.75 and 8 (N=51-110 neurons per calibration point; calibration curve linear region goodness of fit R²=0.999, between pH 7.15-8.0). Images were processed using ImageJ (National Institutes of Health).

Cell health assays. Membrane properties were measured on day 11 in vitro using algorithms built into pClamp10. Cell death was assayed at day 18 in vitro by incubating cultured neurons for 10 minutes in 0.04% Trypan Blue (Gibco) in Tyrode solution for 10 minutes at room temperature, washing with Tyrode solution, and then immediately counting the percentage of neurons stained. In order to limit variability across cell cultures and cell ages, Arch and wild type control neurons were chosen from the same cell cultures and tested on the same day.

Estimations of thresholds for silencing. Whole-cell current-clamped neurons were somatically injected with 5 Hz current pulse trains (15 ms pulse duration, 8 sec train duration) at 200, 350, or 500 pA, with or without yellow light (575 nm) illumination at irradiances of 6, 1.28, or 0.35 mW/mm² for 3 seconds, beginning 2 seconds into the current pulse train. Neurons that did not spike at all with 200 pA current pulses (15 ms pulse duration) were discarded. For all remaining cells, the probability of spiking in the dark, given a 200 pA/15 ms current input, was 84.0±10.5% and 82.8±3.5% for Halo- and Arch-expressing neurons, respectively (p>0.9, N=7-8 neurons each), and the probability of spiking in the dark was ˜100%, given ≧350 pA current inputs.

Virus injection. Under isoflurane anesthesia, 1 μA lentivirus was injected through a craniotomy made in the mouse skull, into the motor cortex (0.62 mm anterior, 0.5 mm lateral, and 0.5 mm deep, relative to bregma), or the sensory cortex (0.02 mm posterior, 3.2 mm lateral, and 2.2 mm deep, relative to bregma). Virus was injected at a rate of 0.1-0.2 μl/min through a cannula connected via polyethylene tubing to a Hamilton syringe, placed in a syringe pump (Harvard Apparatus). The syringe, tubing, and cannula were filled with silicone oil (Sigma). For mice used for in vivo recordings, custom-fabricated plastic headplates were affixed to the skull⁴³, and the craniotomy was protected with agar and dental acrylic.

In vivo physiology, optical neuromodulation, and data analysis. Recordings were made in the cortex of headfixed awake mice 1-2 months after virus injection, using glass microelectrodes of 5-20 MΩ impedance filled with PBS, containing silver/silver-chloride wire electrodes. Signals were amplified with a Multiclamp 700B amplifier and digitized with a Digidata 1440, using pClamp software (Molecular Devices). A 50 mW yellow laser (SDL-593-050T, Shanghai Dream Laser) was coupled to a 200 micron-diameter optical fiber in a fashion as described previously. The laser was controlled via TTL pulses generated through Digidata. Laser light power was measured with an 818-SL photodetector (Newport Co.). An optical fiber was attached to the recording glass electrode, with the tip of the fiber ˜600 μm laterally away from and ˜500 μm above the tip of the electrode (e.g., ˜800 microns from the tip), and guided into the brain with a Siskiyou manipulator at a slow rate of ˜1.5 μm/s to minimize deformation of the cortical surface.

Data was analyzed using MATLAB (Mathworks, Inc.). Spikes were detected and sorted offline using Wave_clus. Neurons suppressed during light were identified by performing a paired t-test, for each neuron, between the baseline firing rate during the 5 second period before light onset vs. during the period of 5 second light illumination, across all trials for that neuron, thresholding at the p<0.05 significance level. Instantaneous firing rate histograms were computed by averaging the instantaneous firing rate for each neuron, across all trials, with a histogram time bin of 20 ms duration. To determine the latency between light onset and the neural response, a 20 ms-long sliding window was swept through the electrophysiology data and the earliest 20 ms period that deviated from baseline firing rate was identified, as assessed by performing a paired t-test for the firing rate during each window vs. during the baseline period, across all trials for each neuron. Latency was defined as the time from light onset to the time at which firing rate was significantly different from baseline for the following 120 ms. The time for after-light suppression to recover back to baseline was calculated similarly.

Histology. Between 2 and 8 weeks after virus injection, mice were perfused through the left cardiac ventricle with ˜20 mL 4% paraformaldehyde in PBS (pH 7.3), and then the brain was removed and sectioned into 120-240 μm coronal sections on a vibratome in ice-cold PBS, and stored in PBS. Slices were mounted with Vectashield solution (Vector Labs), and visualized with a Zeiss LSM 510 confocal microscope using 20× and 63× objective lenses.

Monte Carlo modeling of light propagation. In MATLAB, a Monte Carlo simulation of light scattering and absorption in the brain from light emitted from the end of an optical fiber was performed by dividing a cube of gray matter into a 200×200×200 grid of voxels corresponding to 10 μm×10 μm×10 μm in dimension, using previously-published model parameters and algorithms. Data was interpolated from the literature to obtain a scattering coefficient for yellow (−593 nm) light in brain gray matter of 13 mm⁻¹, and an absorption coefficient of 0.028 mm⁻¹. Since light propagation close to the optical fiber was of interest, before the orientation of photon trajectories is randomized by multiple scattering events, an anisotropic scattering model based upon the Henyey-Greenstein phase function was used, utilizing an anisotropy parameter of 0.89. 5×10⁶ packets of photons were launched in a fiberlike radiation pattern through a model fiber (Optran 0.48 HPCS, Thorlabs) with a numerical aperture of 0.48, and modeled their propagation into the brain based on the algorithm of Wang, L., Jacques, S. L. & Zheng, L. MCML—Monte Carlo modeling of light transport in multi-layered tissues. Computer methods and programs in biomedicine 47, 131-146 (1995). In essence, whenever a photon packet entered a voxel, the program would probabilistically calculate the forecasted traveling distance before the next scattering event. If that traveling distance took the photon packet out of the starting voxel, then the packet would be partially absorbed appropriately for the distance it traveled within the starting voxel, and the process would then restart upon entry of the photon packet into the new voxel. If that traveling distance ended the trip of the photon packet within the starting voxel, then the packet would be absorbed appropriately for the distance it traveled within the starting voxel, and a new direction of packet propagation would be randomly chosen according to the Henyey-Greenstein function. Using this model, a graph was generated that shows the contours at which the light irradiance falls off to various percentages of the irradiance of light at the surface of the optical fiber, for yellow light. The model generally agrees with previous measurements, done in brain slices.

While a preferred embodiment is disclosed, many other implementations will occur to one of ordinary skill in the art and are all within the scope of the invention. Each of the various embodiments described above may be combined with other described embodiments in order to provide multiple features. Furthermore, while the foregoing describes a number of separate embodiments of the apparatus and method of the present invention, what has been described herein is merely illustrative of the application of the principles of the present invention. Other arrangements, methods, modifications, and substitutions by one of ordinary skill in the art are therefore also considered to be within the scope of the present invention, which is not to be limited except by the claims that follow.

What is claimed is:

1. A method for adjusting the voltage potential of cells, subcellular regions, or extracellular regions, the method comprising:
incorporating at least one gene encoding for a light-driven proton pump into at least one target cell, subcellular region, or extracellular region, the proton pump operating to change transmembrane potential in response to a specific wavelength of light; and
causing the expression of the gene by exposing the target cell, subcellular region, or extracellular region to the specific wavelength of light in a manner designed to cause the voltage potential of the target cell, subcellular region, or extracellular region to increase or decrease.

2. The method of claim 1, wherein the proton pump is a microbial rhodopsin that is outwardly rectifying.

3. The method of claim 1, wherein the proton pump is derived from the halorubrum genus of archaeabacteria.

4. The method of claim 3, wherein the proton pump is derived from an organism selected from the group consisting of halorubum strain aus-1, halorubrum strain aus-2, halorubrum sodomense, halorubrum strain BD1, halorubrum strain xz515, halorubrum strain TP009, and halorubrum lacusprofundi.

5. The method of claim 1, wherein the proton pump is derived from an organism selected from the group consisting of leptosphaeria maculans, P. triticirepentis, and S. scelorotorium.

6. The method of claim 1, further comprising the step of increasing or decreasing the voltage potential of the target cell, subcellular region, or extracellular region until it is hyperpolarized.

7. The method of claim 6, wherein the target cell, subcellular region, or extracellular region is a neuron and the hyperpolarization achieves neural silencing.

8. The method of claim 7, further comprising the step of using a plurality of light-activated proton pumps responsive to different wavelengths of light to achieve multi-color neural silencing by the steps of:
expressing each light-activated proton pump in a different population of cells; and
illuminating the cells with different colors of light.

9. The method of claim 2, wherein the microbial rhodopsin is both inwardly and outwardly rectifying at two different wavelengths of light.

10. A method for adjusting the pH of cells, subcellular regions, or extracellular regions, the method comprising:
incorporating at least one gene encoding for a light-driven proton pump into at least one target cell, subcellular region, or extracellular region, the proton pump operating to change cell, subcellular region, or extracellular region pH in response to a specific wavelength of light; and
causing the expression of the gene by exposing the target cell, subcellular region, or extracellular region to the specific wavelength of light in a manner designed to cause the pH of the target cell, subcellular region, or extracellular region to increase or decrease.

11. The method of claim 10, wherein the proton pump is a microbial rhodopsin that is outwardly rectifying.

12. The method of claim 10, wherein the proton pump is derived from the halorubrum genus of archaeabacteria.

13. The method of claim 12, wherein the proton pump is derived from an organism selected from the group consisting of halorubum strain aus-1, halorubrum strain aus-2, halorubrum sodomense, halorubrum strain BD1, halorubrum strain xz515, halorubrum strain TP009, and halorubrum lacusprofundi.

14. The method of claim 10, wherein the proton pump is derived from an organism selected from the group consisting of leptosphaeria maculans, P. triticirepentis, and S. scelorotorium.

15. The method of claim 11, wherein the microbial rhodopsin is both inwardly and outwardly rectifying at two different wavelengths of light.

16. A method for causing cells, subcellular regions, or extracellular regions to release protons as chemical transmitters, the method comprising:
incorporating at least one gene encoding for a light-driven proton pump into at least one target cell, subcellular region, or extracellular region, the proton pump operating to cause proton release in response to a specific wavelength of light; and
causing the expression of the gene by exposing the target cell, subcellular region, or extracellular region to the specific wavelength of light in a manner designed to cause the target cell, subcellular region, or extracellular region to release protons.

17. The method of claim 16, wherein the proton pump is a microbial rhodopsin that is outwardly rectifying.

18. The method of claim 16, wherein the proton pump is derived from the halorubrum genus of archaeabacteria.

19. The method of claim 18, wherein the proton pump is derived from an organism selected from the group consisting of halorubum strain aus-1, halorubrum strain aus-2, halorubrum sodomense, halorubrum strain BD1, halorubrum strain xz515, halorubrum strain TP009, and halorubrum lacusprofundi.

20. The method of claim 16, wherein the proton pump is derived from an organism selected from the group consisting of leptosphaeria maculans, P. triticirepentis, and S. scelorotorium.


SEQUENCE LISTING (17 sequences)

SEQ ID NO: 1 - protein, 258 aa, Halorubrum sodomense
MDPIALQAGYDLLGDGRPETLWLGIGTLLMLIGTFYFLVRGWGVTDKDAREYYAVTILVPGIASAAYLSMFFGIGLTEVTVGGEMLDIYYARYADWLFTTPLLLLDLALLAKVDRVTIGTLVGVDALMIVTGLIGALSHTAIARYSWWLFSTICMIVVLYFLATSLRSAAKERGPEVASTFNTLTALVLVLWTAYPILWIIGTEGAGVVGLGIETLLFMVLDVTAKVGFGFILLRSRAILGDTEAPEPSAGADVSAAD

SEQ ID NO: 2 - DNA, 797 bp, Artificial Sequence
  human codon-optimized DNA sequence; Genbank accession # GU045593; Genbank accession # GU045594 when fused to C-terminal GFP
ggatccgccaccatggaccccatcgctctgcaggctggttacgacctgctgggtgacggcagacctgaaactctgtggctgggcatcggcactctgctgatgctgattggaaccttctactttctggtccgcggatggggagtcaccgataaggatgcccgggaatattacgctgtgactatcctggtgcccggaatcgcatccgccgcatatctgtctatgttctttggtatcgggcttactgaggtgaccgtcgggggcgaaatgttggatatctattatgccaggtacgccgactggctgtttaccaccccacttctgctgctggatctggcccttctcgctaaggtggatcgggtgaccatcggcaccctggtgggtgtggacgccctgatgatcgtcactggcctcatcggagccttgagccacacggccatagccagatacagttggtggttgttctctacaatttgcatgatagtggtgctctattttctggctacatccctgcgatctgctgcaaaggagcggggccccgaggtggcatctacctttaacaccctgacagctctggtcttggtgctgtggaccgcttaccctatcctgtggatcataggcactgagggcgctggcgtggtgggcctgggcatcgaaactctgctgtttatggtgttggacgtgactgccaaggtcggctttggctttatcctgttgagatcccgggctattctgggcgacaccgaggcaccagaacccagtgccggtgccgatgtcagtgccgccgactagcgaccggt

SEQ ID NO: 3 - protein, 248 aa, Halorubrum strain TP009
MDPIALQAGYDLLGDGRPETLWLGIGTLLMLIGTFYFIVKGWGVTDKEAREYYSITILVPGIASAAYLSMFFGIGLTEVTVAGEVLDIYYARYADWLFTTPLLLLDLALLAKVDRVSIGTLVGVDALMIVTGLIGALSHTPLARYSWWLFSTICMIVVLYFLATSLRAAAKERGPEVASTFNTLTALVLVLWTAYPILWIIGTEGAGVVGLGIETLLFMVLDVTAKVGFGFILLRSRAILGDTEAPEP

SEQ ID NO: 4 - DNA, 764 bp, Artificial Sequence
  mammalian codon-optimized DNA sequence
ggatccgccaccatggaccccatcgctctgcaggcaggatacgacctgctgggcgacggaaggccagagaccctgtggctgggaatcggaaccctgctgatgctgatcggcaccttctacttcatcgtgaagggctggggcgtgaccgacaaggaggccagggagtactacagcatcacaatcctggtgcccggcatcgccagcgccgcctacctgagcatgttcttcggcatcggcctgaccgaggtgaccgtggccggcgaggtgctggacatctactacgccagatacgccgactggctgttcaccacccctttgcttctgctcgacctggccctgctggctaaggtggacagggtgagcatcggaaccctggtgggagtggacgccctgatgatcgtgaccggcctgatcggcgccctgagccacaccccactggctaggtacagctggtggctgttcagcaccatctgcatgatcgtggtgctgtacttcctggctaccagcctgagggctgctgctaaggagaggggaccagaggtggctagcaccttcaacaccctgaccgccctggtgctggtgctgtggaccgcctaccccatcctgtggatcatcggaaccgagggagctggagtggtgggactgggaatcgagaccctgctgttcatggtgctggacgtgaccgccaaagtgggcttcggcttcatcctgctgaggagcagggccatcctgggcgacaccgaggcccccgagccccgaccggt

SEQ ID NO: 5 - protein, 313 aa, Leptosphaeria maculans
MIVDQFEEVLMKTSQLFPLPTATQSAQPTHVAPVPTVLPDTPIYETVGDSGSKTLWVVFVLMLIASAAFTALSWKIPVNRRLYHVITTIITLTAALSYFAMATGHGVALNKIVIRTQHDHVPDTYETVYRQVYYARYIDWAITTPLLLLDLGLLAGMSGAHIFMAIVADLIMVLTGLFAAFGSEGTPQKWGWYTIACIAYIFVVWHLVLNGGANARVKGEKLRSFFVAIGAYTLILWTAYPIVWGLADGARKIGVDGEIIAYAVLDVLAKGVFGAWLLVTHANLRESDVELNGFWANGLNREGAIRIGEDDGA

SEQ ID NO: 6 - DNA, 959 bp, Artificial Sequence
  mammalian codon-optimized DNA sequence; Genbank accession # GU045595; Genbank accession # GU045596 when fused to C-terminal GFP
ggatccgccaccatgatcgtggaccagttcgaggaggtgctgatgaagaccagccagctgttcccactgccaaccgctacccagagcgcccagccaacccacgtggcccccgtgccaaccgtgctgcccgacacccccatctacgagaccgtgggcgacagcggcagcaagaccctgtgggtggtgttcgtgctgatgctgatcgccagcgccgccttcaccgccctgagctggaagatccccgtgaacaggaggctgtaccacgtgatcaccaccatcatcaccctgaccgccgccctgagctacttcgctatggctaccggccacggagtggccctgaacaagatcgtgatcaggacccagcacgaccacgtgcccgacacctacgagaccgtgtaccgacaggtgtactacgccaggtacatcgactgggctatcaccaccccactgctgctgctggacctgggactgctggctggaatgagcggagcccacatcttcatggccatcgtggctgacctgatcatggtgctgaccggcctgttcgctgctttcggcagcgagggaaccccacagaagtggggatggtacaccatcgcctgcatcgcctacatcttcgtggtgtggcacctggtgctgaacggcggcgccaacgccagggtgaagggcgagaagctgaggagcttcttcgtggccatcggagcttacaccctgatcctgtggaccgcttacccaatcgtgtggggactggctgacggagctaggaagatcggagtggacggagagatcatcgcttacgctgtgctggacgtgctggctaagggagtgttcggagcttggctgctggtgacccacgccaacctgagggagagcgacgtggagctgaacggcttctgggccaacggcctgaacagggagggcgccatcaggatcggcgaggacgacggcgcccgaccggt

SEQ ID NO: 7 - protein, 29 aa, Artificial Sequence
  N-terminal prolactin endoplasmic sorting sequence (denoted as ′PRL′)
MDSKGSSQKGSRLLLLLVVSNLLLCQVVS

SEQ ID NO: 8 - DNA, 96 bp, Artificial Sequence
  Synthetic oligonucleotide
gacagcaaaggttcgtcgcagaaagggtcccgcctgctcctgctgctggtggtgtcaaatctactcttgtgccagggtgtggtctccacccccgtc

SEQ ID NO: 9 - protein, 21 aa, Artificial Sequence
  MHC class I antigen signal sequence
MVPCTLLLLLAAALAPTQTRA

SEQ ID NO: 10 - DNA, 60 bp, Artificial Sequence
  Synthetic oligonucleotide
gtcccgtgcacgctgctcctgctgttggcagccgccctggctccgactcagacgcgggcc

SEQ ID NO: 11 - protein, 28 aa, Homo sapiens
MSVLTPLLLRGLTGSARRLPVPRAKIHS

SEQ ID NO: 12 - DNA, 87 bp, Artificial Sequence
  Synthetic oligonucleotide
atgtccgtcctgacgccgctgctgctgcggggcttgacaggctcggcccggcggctcccagtgccgcgcgccaagatccattcgttg

SEQ ID NO: 13 - protein, 19 aa, Homo sapiens
MRGTPLLLVVSLSFSLLQD

SEQ ID NO: 14 - DNA, 54 bp, Unknown
  Unknown organism oligonucleotide
atgaggggtacgcccctgctcctcgtcgtctctctgttctctctgcttcaggac

SEQ ID NO: 15 - protein, 7 aa, Unknown
  ER2 sequence
FCYENEV

SEQ ID NO: 16 - DNA, 21 bp, Halorubrum sodomense
ttctgctacgagaatgaagtg

SEQ ID NO: 17 - protein, 9 aa, Unknown
  C-terminal golgi export sequence from bovine rhodopsin
TETSQVAPA